(19) United States
(12) Patent Application Publication
(10) Pub. No.: US 2026/0256963 A1
(43) Pub. Date: Sep. 3, 2026

(54) Combined Therapy with Nanoparticles and Radiopharmaceuticals

(71) Applicants: Université de Montpellier, Montpellier (FR); Université Claude Bernard Lyon 1, Villeurbanne (FR); INSTITUT REGIONAL DU CANCER DE MONTPELLIER ICM, Montpellier Cedex 5 (FR); NH TherAguix, Meylan (FR); INSTITUT NATIONAL DE LA SANTE ET DE LA RECHERCHE MEDICALE, Paris (FR); Centre national de la recherche scientifique, Paris (FR)

(72) Inventors: Jean-Pierre POUGET, Notre Dame de Londres (FR); Julie CONSTANZO, Grabels (FR); Clara DIAZ GARCIA-PRADA, Montpellier (FR); Léna CARMES, Lyon (FR); Olivier TILLEMENT, Fontaines Saint Martin (FR); François LUX, Villeurbanne (FR)

(21) Appl. No.: 18/993,337

(22) PCT Filed: Jul. 12, 2023
(86) PCT No.: PCT/EP2023/069409

§ 371 Date: Jan. 10, 2025

(30) Foreign Application Priority Data
Jul. 13, 2022 (EP) 22306057.5

(51) Int. Cl. A61K 51/10; A61K 9/51; A61K 33/24; A61P 35/00

(52) U.S. Cl. CPC A61K 51/1096; A61K 9/5146; A61K 33/24; A61K 51/1051; A61P 35/00

(57) ABSTRACT

The present invention relates to a high-Z element containing nanoparticles for use in a method of treating a tumor by radiopharmaceutical therapy, in a subject in need thereof, the method comprising a combined administration of an efficient amount of said high-Z element containing nanoparticles and of an efficient amount of a radionuclide containing therapeutic radiopharmaceutical, wherein the high-Z element containing nanoparticles contain an element with an atomic Z number higher than 40, preferably higher than 50, and wherein said nanoparticles have a mean hydrodynamic diameter of 20 nm or less, for example between 1 and 10 nm, preferably between 2 and 8 nm.

### BACKGROUND

The principle of radiotherapy is to induce unrepairable DNA lesions in tumor cells, leading to cell death. Half of patients with cancer receive conventional external radiotherapy (X-RT) that irradiates the tumor from outside the body. This approach is suitable for treating localized tumors or oligometastases, but it cannot be used generally for diffuse or metastatic disease because of unacceptable irradiation to healthy tissues. With several compounds recently approved by FDA and EMA, radiopharmaceutical therapy also termed targeted radionuclide therapy (TRT) has emerged as a safe and effective systemic therapeutic modality to irradiate all tumor sites (Sgouros et al., 2020).

In TRT, radiolabeled cancer-binding molecules (e.g. antibody, peptides) are injected in patients. After circulating in the bloodstream, they recognize, bind to, and locally irradiate tumor cells. TRT allows using β-particles and also the highly potent α-particles or Auger electron emitters (AEEs). Like the X-rays used in X-RT, β-particles are low linear energy transfer (LET) particles and produce “simple DNA lesions”, such as single- and double-strand breaks (SSBs, DSBs) or base damage (e.g. thymidine glycols). Conversely, α-particles and to a lower extent AEEs are high LET particles that produce unrepairable complex or clustered DNA lesions, making them an attractive candidate to overcome radiation resistance. Unlike X-RT, TRT irradiation is protracted, delivered at low dose and low dose rate, thus reducing the side effects in bone marrow and circulating blood cells. For these reasons, radiobiology of X-RT cannot be directly extrapolated to TRT (Pouget et al., 2011; Pouget et al., 2021).

Ovarian Cancer (OC) is the most lethal gynecological malignancy nowadays, and the 8^{th} most-frequent cause of cancer-related death among women worldwide. Histologically, 90% of OC originate from a malignant transformation of an epithelial cell. In this context, the most aggressive form of OC arises from the epithelium of the fallopian tubes, known as High Grade Serous Ovarian Carcinoma (HGSOC). The disease progression without clinical signs or symptoms in most of cases, leads to a late-stage diagnosis (stage III/IV) when it has spread into the peritoneal cavity under the form of peritoneal carcinomatosis (PC). Treatment of PC consists of cytoreductive surgery to remove the macroscopic disease, followed by intraperitoneal adjuvant platinum-based chemotherapy. Although many women respond to this therapeutic approach, disease recurs in 70-90% of cases, remaining localized in the peritoneal cavity.

In the 1980s, Sugarbaker proposed the combination of cytoreductive surgery and hyperthermic intraperitoneal chemotherapy to treat the residual disease in selected patients with h peritoneal carcinomatosis (Sugarbaker, 2009). Hyperthermic intraperitoneal chemotherapy increases survival in patients with primary recurrent disease and is today used as first- or second-line therapy. However, because morbidity has been one of the main drawbacks of hyperthermic intraperitoneal chemotherapy, it is not recommended outside clinical trials (Goodman et al., 2016). Consequently, there is an urgent need to suppress the residual disease, responsible for patient's relapse. For this type of metastatic and diffuse diseases, conventional radiotherapy cannot be applied because of the high risk of damage in the surrounding healthy tissues. TRT-based strategies using radiolabeled antibodies specifically directed against tumor nodules gives new treatment opportunities for treating HGSOC (Pouget et al., 2015).

Several studies on rodents have shown that radioimmunotherapy (RIT) is an efficient adjuvant after cytoreductive surgery for peritoneal carcinomatosis (Koppe et al., 2005; Aarts et al., 2007; Aarts et al., 2008; Müller et al., 2012; Seidl et al. 2011; Milenic et al., 2004; Andersson et al., 2003; Elgqvist et al., 2005) Several antibodies (against MUC1, CA-125, TAG72, and gp38) have been conjugated to 4 β-emitting radionuclides for IP RIT in patients with ovarian cancer (Pouget et al., 2011). Based on previous encouraging results (Meredith et al., 2007; Alvarez et al., 2002; Epenetos et al., 2002; Hird et al., 1993) a phase III randomized multicenter study was undertaken (Verheijen et al., 2006) in which the efficiency of conventional chemotherapy was compared with IP injection of ⁹⁰Y-labeled HMGF1 murine mAb (anti-MUC1). However, no improvement in survival was observed after RIT, although peritoneal recurrence was significantly delayed.

Although TRT-based strategies are very promising, the therapeutic index still needs to be improved. In particular, adverse side effects on healthy tissues (e.g., the bone marrow) limit dose escalation. This need is even more pronounced in situations where tumors, like ovarian, are radioresistant.

Thus, there is still a need to overcome the drawbacks of the prior art and to provide improved TRT-based protocols.

The present disclosure follows in part from the surprising findings by the inventors that the nanoparticles according to the present disclosure, when used in combination with therapeutic radiopharmaceuticals compounds in a TRT protocol, lead to an increased efficacy of TRT. Surprisingly, the combined therapy allows a tumor colocalization of the nanoparticles with the therapeutic radiopharmaceutical compounds sufficient to result in potentiating the effect of the therapeutic radiopharmaceutical compounds.

Such nanoparticles can thus be used in combination with therapeutic radiopharmaceutical to enhance the effectiveness of targeted radiotherapy, or to maintain its efficacy while decreasing the dose of therapeutic radiopharmaceutical to be administered.

### BRIEF DESCRIPTION

Accordingly, an embodiment E1 of the present disclosure relates a high-Z element containing nanoparticles for use in a method of treating a tumor by radiopharmaceutical therapy, in a subject in need thereof, the method comprising a combined administration of an efficient amount of said high-Z element containing nanoparticles and of an efficient amount of a radionuclide containing therapeutic radiopharmaceutical, wherein the high-Z element containing nanoparticles contain an element with an atomic Z number higher than 40, preferably higher than 50, and wherein said nanoparticles have a mean hydrodynamic diameter of 20 nm or less, for example between 1 and 10 nm, preferably between 2 and 8 nm.

An embodiment E2 of the present disclosure relates to the nanoparticles for use in a method according to embodiment E1, wherein the nanoparticles enhance the therapeutic efficacy of said radiopharmaceutical.

An embodiment E3 of the present disclosure relates to the nanoparticles for use according to any of embodiments E1 or E2, wherein the high-Z element is selected among the heavy metals, and more preferably, Au, Ag, Pt, Pd, Sn, Ta, Zr, Tb, Tm, Ce, Dy, Er, Eu, La, Nd, Pr, Lu, Yb, Bi, Hf, Ho, Pm, Sm, In, and Gd, and mixtures thereof.

An embodiment E4 of the present disclosure relates to the nanoparticles for use according to any of embodiments E1 to E3, wherein the radionuclide is selected from ¹⁷⁷Lu, ¹⁶¹Tb, ¹⁸⁶Re, ¹³¹I, ⁹⁰Y, ²²⁵Ac/²¹³Bi, ²²³Ra, ²¹²Pb/²¹²Bi, ²²⁷Th, ²¹¹At, ₉₇Ru, ¹⁰³Pd, ⁶⁷Ga, ^{195m}Pt, ^{193m}Pt, ¹²⁵I, ¹¹¹In, and mixtures thereof.

An embodiment E5 of the present disclosure relates the nanoparticles for use according to any of Embodiment E1 to E3, wherein the efficient amount of radiopharmaceutical is comprised between 0.5.MBq and 100 GBq, preferably between 1MBq and 100 GBq.

An embodiment E6 of the present disclosure relates to the nanoparticles for use according to any of embodiments E1 to E4, wherein the radionuclide is linked to a cancer-targeting moiety.

An embodiment E7 of the present disclosure relates to the nanoparticles for use according to embodiment E5, wherein the cancer-targeting moiety is an antibody, a peptide or a small-molecule ligand.

An embodiment E8 of the present disclosure relates to the nanoparticles for use according to any of embodiment E6 or E7, wherein the cancer-targeting moiety is selected from
an anti-HER2 antibody such as trastuzumab, pertuzumab, or ibritumomab (also referred to as ibritumomab tiuxetan, commercialized under the trademark Zevalin®), an anti-EGFR antibody such as cetuximab or panitumumab, an anti-CD20 antibody such as rituximab or zevalin, an anti-CD33 antibody such as lintuzumab, an anti-CD37 antibody such as Otlertuzumab (TRU-016), mAB 37.1 (BI 836826) or IMGN529 (K7153A-DM1), an anti-AMHRII antibody such as murlentamab, or an anti-TYRP1/gp75 antibody such as IMC-20D7S,
a somatostatin analog such as Octreotide (DOTATOC) or Octreotate (DOTATATE), or
a PSMA small-molecule ligand such as 617 ligand, I&T ligand, R2 ligand or MIP-1095 ligand.

An embodiment E9 of the present disclosure relates to the nanoparticles for use according to any of embodiments E1 to E8, wherein the therapeutic radiopharmaceutical is selected from an ¹⁷⁷Lu-anti-HER2 antibody such as ¹⁷⁷Lu-trastuzumab, an ¹⁷⁷Lu-somatostatin analog such as ¹⁷⁷Lu-dotatate, a ¹⁷⁷Lu-PSMA ligand, an ⁹⁰Y-anti-CD20 antibody such as ⁹⁰Y-rituximab or ⁹⁰Y-ibritumomab, an ²¹²Pb-anti-HER2 antibody, an ¹⁷⁷Lu-anti CD37 antibody.

An embodiment E10 of the present disclosure relates to the nanoparticles for use according to any of embodiments E1 to E4, wherein the therapeutic radiopharmaceutical consists of ¹³¹I or ²²³Ra.

An embodiment E11 of the present disclosure relates to the nanoparticles for use according to any of embodiments E1 to E10, wherein the nanoparticles are administered to the subject in a fractionated dose regimen.

An embodiment E12 of the present disclosure relates to the nanoparticles for use according to any of embodiments E1 to E11, wherein the fractionated dose regimen of nanoparticles comprises 2 to 10 fractionated doses of nanoparticles for each dose of therapeutic radiopharmaceutical administered to the subject.

An embodiment E13 of the present disclosure relates to the nanoparticles for use according to any of embodiment E1 to E12, wherein the subject is a subject that cannot receive the standard effective dose of a radiopharmaceutical therapy.

An embodiment E14 of the present disclosure relates to the nanoparticles for use according to any of embodiment E1 to E13, wherein the tumor is a radioresistant tumor.

An embodiment E15 of the present disclosure relates to the nanoparticles for use according to any of embodiments E1 to E14, wherein the tumor is selected from peritoneal tumors including primary peritoneal tumors and secondary peritoneal tumors, neuroendocrine tumors including gastroenteropancreatic neuroendocrine tumors and pheochromocytoma or paragangliomas (PPGLs), prostate tumor, neuroblastoma, meningioma, lymphoma, Merkel cell carcinoma, breast tumor, renal cell tumor, and salivary gland carcinoma.

### LEGENDS TO THE FIGURES

FIG. 1 illustrates AGuIX® fractionated regimens assayed in Example 1. Regimen 1 (5×4 mg): mice received a single injection of 4 mg AGuIX in 200 μl saline solution for 5 consecutive days. Regimen 2 (10×2 mg): mice received two injections of 2 mg AGuIX in 200 μl saline solution per day (separated with a 6 h time lapse) for 5 consecutive days. Regimen 3 (4×5 mg): mice received two injections of 5 mg AGuIX in 200 μl saline solution per day (separated with a 6 h time lapse) 24 h and 72 h post-TRT.

FIG. 2 represents the total tumor mass (mg) in SK-OV-3-luc xenografts 4 weeks post-treatment with 10 MBq with or without AGuIX®. The results show that a 10 MBq activity is too effective to appreciate AGuIX® radiosensitizing effect. TZ: Trastuzumab. 10 MBq: 10MBq ¹⁷⁷Lu-Trastuzumab.

FIG. 3 represents the total tumor mass (mg) in SK-OV-3-luc xenografts 4 weeks post-treatment with 2.5 or 5 MBq with or without AGuIX®. The results show that an activity of 5MBq ¹⁷⁷Lu-Trastuzumab associated to 10 mg AGuIX® showed a radiosensitizing trend. TZ: Trastuzumab. 2.5 MBq: 2.5 MBq ¹⁷⁷Lu-Trastuzumab. 5 MBq: 5 MBq ¹⁷⁷Lu-Trastuzumab.

FIG. 4 illustrates the Gadolinium (Gd) quantification by ICP-MS in tumor nodules, heart, blood, liver and kidneys in mice treated with 5MBq ¹⁷⁷Lu-Trastuzumab associated to 10 mg AGuIX®

FIG. 5 illustrates the total tumor mass (mg) in SK-OV-3-luc xenografts 4 weeks post-treatment with 5 MBq with AGuIX® according to Regimen 1, Regimen 2 or Regimen 3. The results show a significant difference between TRT (5MBq) and the fractionated Regimen 3 (FIG. 5A) and the RECIST criteria evaluation between 5 MBq and 5 MBq+Regimen 3 (FIG. 5B).

FIG. 6 illustrates A) Clonogenic cell survival of SK-OV-3 and A431 cells exposed to ¹⁷⁷Lu-Trastuzumab at 0.5, 1, 2 and 4 MBq/mL±10 mg/ml of AGuIX®. B) Clonogenic cell survival of B16F10 cells exposed to 4 MBq/mL ¹²⁵I-TA99±1 mg/ml of AGuIX®. C) Clonogenic cell survival of MiaPaca2 cells exposed to 0.8, 2 and 4 MBq/mL of LUTATHERA®±1, 5 and 10 mg/ml of AGuIX®.

FIG. 7 illustrates the biodistribution of ¹⁷⁷Lu-labeled Trastuzumab. Athymic female Swiss nude mice were intraperitoneally (IP) xenografted with 3×106 SK-OV-3-luc cells. 14 days later, mice were IP injected with ¹⁷⁷Lu-labeled Trastuzumab. Tumors and organs were collected, weighed and radioactivity uptake measured by γ-counting. For each organ or tumor, percentage of injected activity per gram of tissue (% IA/g) were plotted.

FIG. 8 shows the Kaplan-Meier survival and mean tumor absorbed dose of mice bearing intraperitoneal SK-OV-3-luc tumor cell xenografts that received a single intraperitoneal injection of NaCl, 25 μg trastuzumab+AGuiX® (2×5 mg per day, 24 h and 72 h post-trastuzumab), 5MBq ¹⁷⁷Lu-trastuzumab, or 5 MBq of ¹⁷⁷Lu-trastuzumab+AGuiX® (2×5 mg per day, 24 h and 72 h post-TRT).

FIG. 9 illustrates the clonogenic survival of SK-OV-3-luc, A-431 and OVCAR-3 cells incubated with ¹⁷⁷Lu-trastuzumab (0-4 MBq/mL) with/without 10 mg/mL AGuiX® for 18 h in the presence or not of deferiprone (DFP). Results are the mean±SD of three independent experiments performed in triplicate; * p<0.05, ** p <0.01, *** p<0.001, ns: not significant (Mann-Whitney t test) compared with cells treated with ¹⁷⁷Lu-trastuzumab. These data show that ferroptosis is potentially involved in the radiosensitizing effects of high-Z NPs according to the invention such as AGuiX®.

FIG. 10 shows TEM micrographs of SKOV3 cells 48 h after treatment with 1 MBq/mL of ¹⁷⁷Lu-Trastuzumab+10 mg/mL AGuIX® in the presence of the iron chelator Deferiprone (DFP). Cytoplasm dissolution/necrosis-like features with arrows. The left panel shows the outcome of TRT+NP treatment 48 h post-incubation and the right panel shows the same treatment in the presence of DFP. Cytoplasmic vacuolization (arrow) is abolished in the presence of the iron chelator. Altogether, TRT+AGuIX® combination lead to dramatic ultrastructural modifications observed by TEM, characterized by a massive cytoplasmic vacuolization followed by an apparent cytoplasmic dissolution and the accumulation of autophagosomes and damaged undigested cell components. Iron chelation showed to increase TRT+NP-treated cell survival, recover lysosomal integrity, and reverse ultrastructural modifications.

### DETAILED DESCRIPTION

The present disclosure follows in part from the surprising findings as shown by the inventors that certain nanoparticles with radiosensitizing properties colocalize with therapeutic radiopharmaceutical compound and substantially increase the efficacy of TRT.

Without wishing to be bound by any particular theory, it is believed that the advantageous effect of the method of treatments of the present disclosure are linked to at least the following two features of these nanoparticles:
(i) they contain high-Z elements, typically complexes of high-Z cations with radiosensitizing properties,
(ii) they have a small mean hydrodynamic diameter allowing a passive targeting to cancer cells.

In present disclosure, “passive targeting to cancer cells” and “passive targeting” refer to the phenomena of having the nanoparticles of the invention accumulating in tumor tissues although not being functionalized for this purpose, in particular not being linked to any cancer-targeting moiety.

Without wishing to be bound by any theory, the inventors believe that this phenomena of having the high-Z element containing nanoparticles according to the invention exerting a passive targeting to cancer cells could possibly be explained at least in part by an enhanced permeability and retention (EPR) effect. EPR effect is a phenomena by which certain molecules, macromolecular compounds or nanoparticles tend to accumulate in tumor tissue. This might be due at least in part to the fact that the endothelial cells of the vessels that irrigate the tumors are somewhat looser, so that circulating objects diffuse more easily, and/or to the fact that tumors are less well drained.

The inventors have shown that, surprisingly, the nanoparticles of the invention, although not being functionalized to target the nanoparticles to specific tissues, and thus reaching the tumor tissue only due to passive targeting, result in potentiating the effect of the therapeutic radiopharmaceutical.

Without wishing to be bound by any theory, the inventors believe that efficacy of the combined administration is due tumor colocalization with the therapeutic radiopharmaceutical compounds and radiosensitizing effect of the high-Z element containing nanoparticles upon the radionuclide containing therapeutic radiopharmaceutical, and that such radiosensitizing effect is mediated by ferroptosis. This aspect is described in more details below.

In the present disclosure, the term “radiosensitizing” would be readily understood by one of ordinary skill in the art and generally refers to the process of increasing the sensitivity of the cancer cells to radiation therapy (e.g., photon radiation, electron radiation, proton radiation, alpha radiation, heavy ion radiation). Said high-Z element as used herein is an element with an atomic Z number higher than 40, for example higher than 50.

In specific embodiments, said high-Z element is selected among the heavy metals, and more preferably, Au, Ag, Pt, Pd, Sn, Ta, Zr, Tb, Tm, Ce, Dy, Er, Eu, La, Nd, Pr, Lu, Yb, Bi, Hf, Ho, Pm, Sm, In, and Gd, and mixtures thereof.

The high-Z elements are preferably cationic elements, either comprised in the nanoparticles as oxide and/or chalcogenide or halide or as complexes with chelating agents, such as organic chelating agents.

The size distribution of the nanoparticles is, for example, measured using a commercial particle sizer, such as a Malvern Zetasizer Nano-S particle sizer based on PCS (Photon Correlation Spectroscopy).

For the purposes of the present disclosure, the term “mean hydrodynamic diameter” or “mean diameter” is intended to mean the harmonic mean of the diameters of the particles. A method for measuring this parameter is also described in standard ISO 13321:1996.

Nanoparticles with a mean hydrodynamic diameter for example below 20 nm, in particular between 1 and 10 nm, and even more preferably between 1 and 8 nm or for example between 2 and 8 nm, or typically around 5 nm, are suitable for the methods disclosed herein. In particular, they have been shown to provide excellent passive targeting in tumors, after intravenous injection, and a rapid renal elimination (and therefore low toxicity).

In an embodiment, the nanoparticles comprise more than 10% by weight of high Z-element, and preferably less than 50% by weight of high Z-element, in % relative to the total weight of the nanoparticles.

In a particular embodiment the nanoparticles comprise between 10 and 50%, preferably between 10 and 20% of Gd, for example about 15%+1% by weight of Gd, in % relative to the total weight of the nanoparticles.

In an embodiment, said nanoparticles include at least 50% by weight of gadolinium (Gd), of dysprosium (Dy), of lutetium (Lu), of bismuth (Bi) or of holmium (Ho), or mixtures thereof, (relative to the total weight of high-Z elements in the nanoparticles), for example at least 50% by weight of gadolinium, as high-Z elements in the nanoparticles.

In a particularly preferred embodiment, said nanoparticle for use in the method of the present disclosure is a gadolinium-based nanoparticle.

In specific embodiments, said high-Z elements are cationic elements complexed with organic chelating agents, for example selected from chelating agents with carboxylic acid, amine, thiol, or phosphonate groups.

In preferred embodiment, the nanoparticles further comprise a biocompatible coating in addition to the high-Z element, and, optionally, the chelating agents. Agent suitable for such biocompatible includes without limitation biocompatible polymers, such as polyethylene glycol, polyethyleneoxide, polyacrylamide, biopolymers, polysaccharides, or polysiloxane.

In particular embodiments, the nanoparticles are chosen such that they have a relaxivity r1 of between 10 and 5000 mM⁻¹·s⁻¹ (at 37° C. and 1.4 T) and/or a Gd weight ratio of at least 5%, for example between 5% and 30%.

In one specific embodiment, said nanoparticles with a very small hydrodynamic diameter, for example between 1 and 10 nm, preferably between 2 and 8 nm, are nanoparticles comprising chelates of high-Z elements, for example chelates of rare earth elements. In certain embodiments, said nanoparticles comprise chelates of gadolinium or bismuth.

In specific embodiments which may be combined with any of the previous embodiments, said high-Z element containing nanoparticles comprise:
polyorganosiloxane,
chelating agents covalently bound to said polyorganosiloxane,
high-Z elements complexed by the chelating agents.

As used herein, the term “chelating agent” refers to one or more chemical moieties capable of complexing one or more metal ions.

Exemplary chelating agents include, but not limited to, 1,4,7-triazacyclononanetriacetic acid (NOTA), 1,4,7,10-tetraazacyclododecane-1,4,7,10-tetraacetic acid (DOTA), 1,4,7-triazacyclononane-l-glutaric acid-4,7-diacetic acid (NODAGA), ethylene diamine tetra-acetic acid (EDTA), diethylene triaminepentaacetic acid (DTPA), cyclohexyl-1,2-diaminetetraacetic acid (CDTA), ethyleneglycol-0,0′-bis(2-aminoethyl)-N, N,N′, N′-tetraacetic acid (EGTA), N,N-bis(hydroxybenzyl)-ethylenediamine-N,N′-diacetic acid (HBED), triethylene tetramine hexaacetic acid (TTHA), hydroxyethyidiamine tnacetic acid (HEDTA), 1,4,8,11-tetraazacyclotetradecane-N,N′,N″, N′″-tetraacetic acid (TETA), and 1,4,7,10-tetraaza-1,4,7,10-tetra-(2-carbamoyl methyl)-cyclododecane (TCMC) and 1,4,7,10-tetraazacyclododececane, 1-(glutaric acid)-4,7,10-triacetic acid (DOTAGA), desferrioxamine (DFO).

In preferred embodiments, said chelating agent is selected among the following:

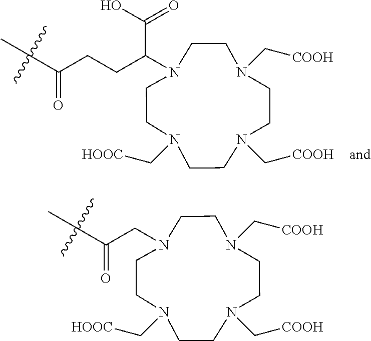 [SMILES: *C(=O)CCC(C(=O)O)N1CCN(CC(=O)O)CCN(CC(=O)O)CCN(CC(=O)O)CC1 ; *C(=O)CN1CCN(CC(=O)O)CCN(CC(=O)O)CCN(CC(=O)O)CC1]

wherein the wavy bond indicates the bond connecting the chelating agent to a linking group of a biocompatible coating forming the nanoparticle.

In a specific embodiment, that may be preferably combined with the previous embodiment, said chelates of rare earth element are chelates of gadolinium and/or bismuth, preferably DOTA or DOTAGA chelating Gd³⁺ and/or Bi³⁺.

In specific and preferred embodiments, the ratio of high-Z element per nanoparticle, for example the ratio of rare earth elements, e.g. gadolinium (optionally as chelated with DOTAGA) per nanoparticle, is between 3 and 100, preferably between 5 and 50, for example between 5 and 20, typically around 10. With such ratio, the nanoparticles have excellent relaxivity and contrast enhancement properties for MR imaging, even when used with MR-Linac with low magnetic field strength, such as 0.35 T or 0.5 T MR-Linac.

In specific embodiments, the hybrid nanoparticles are of core-shell type. Nanoparticles of core-shell type, based on a core consisting of a rare earth oxide and of an optionally functionalized polyorganosiloxane matrix are known (see in particular WO 2005/088314, WO 2009/053644).

The nanoparticles may further be functionalized with molecules which allow targeting of the nanoparticles to specific tissues. Said agents can be coupled to the nanoparticle by covalent couplings, or trapped by non-covalent bonding, for example by encapsulation or hydrophilic/hydrophobic interaction or using a chelating agent.

In one specific embodiment, use is made of hybrid nanoparticles comprising:
a polyorganosiloxane (POS or PS) matrix including, rare earth cations Mⁿ⁺, n being an integer between 2 and 4, optionally partly in the form of a metal oxide and/or oxyhydroxide, optionally associated with doping cations D^{m+}, m being an integer between 2 and 6, D preferably being a rare earth metal other than M, an actinide and/or a transition element;
a chelate covalently bound to the POS via a covalent bond —Si—C—,
the Mⁿ⁺ cations and, where appropriate, D^{m+} cations being complexed by the chelates.

In a preferred embodiment, the nanoparticles are not functionalized with molecules which allow targeting of the nanoparticles to specific tissues, in particular to tumor.

In this embodiment, the nanoparticles reach the tumor only due to passive targeting, as detailed below. In the case of a structure of core-shell type, the POS matrix forms the superficial layer surrounding the metal cation-based core. Its thickness can range from 0.5 to 10 nm, and can represent from 25% to 75% of the total volume.

The POS matrix acts as protection for the core with respect to the external medium (in particular protection against hydrolysis) and it optimizes the properties of the contrast agents (luminescence, for example). It also allows the functionalization of the nanoparticle, via the grafting of chelating agents and of targeting molecules.

### Ultrafine Nanoparticles for Use in the Methods of Treatment of the Disclosure

In a particularly preferred embodiment, said nanoparticles are gadolinium-chelated polysiloxane nanoparticles of the following formula

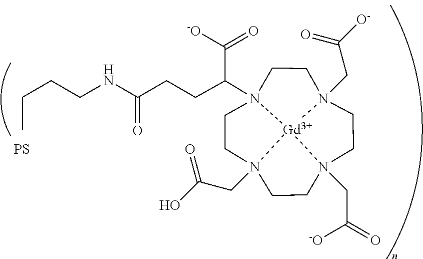

wherein PS is a matrix of polysiloxane, and wherein n is comprised between 5 and 50, typically 5 and 20, and wherein the hydrodynamic diameter is comprised between 1 and 10 nm, for example between 2 and 8 nm, typically about 5 nm.

More specifically, said gadolinium-chelated polysiloxane nanoparticle as described in the above formula is an AGuIX ultrafine nanoparticles as described in the next section.

Such ultrafine nanoparticles that can be used according to the methods of the disclosure may be obtained or obtainable by a top-down synthesis route comprising the steps of:
a. obtaining a metal (M) oxide core, wherein M is a high-Z element as described previously, preferably gadolinium,
b. adding a polysiloxane shell around the M oxide core, for example via a sol gel process,
c. grafting a chelating agent to the POS shell, so that the chelating agent is bound to said POS shell by an —Si—C— covalent bond, thereby obtaining a core-shell precursor nanoparticle, and,
d. transferring the core-shell precursor nanoparticle in an aqueous solution for dissolution of the metal oxide core and purifying,
wherein the grafted agent is in sufficient amount to complex the cationic form of (M), and wherein the mean hydrodynamic diameter of the resulting ultrafine nanoparticle after dissolution of the core is less than 10 nm, for example, between 1 and 10 nm, typically less than 8 nm, for example between 2 and 8 nm.

In preferred embodiments with complete dissolution of the metal oxide core, these nanoparticles obtained according to the method described above do not comprise a core of metal oxide encapsulated by at least one coating. More details regarding the synthesis of these nanoparticles are given hereafter.

This top-down synthesis method results in observed sizes typically of between 1 and 8 nm, more specifically between 2 and 8 nm. The term then used herein is ultrafine nanoparticles.

Alternatively, another “one-pot” synthesis method is described hereafter to prepare said ultrafine nanoparticles with a mean diameter less than 10 nm, for example, between 1 and 8 nm, typically between 2 and 6 nm.

Further details regarding these ultrafine or core-free nanoparticles, the processes for synthesizing them and their uses are described in patent application WO2011/135101, WO2018/224684 or WO2019/008040, which is incorporated by way of reference.

### Process for Obtaining Preferred Embodiments of Nanoparticles for Use in the Methods of Treatment of to the Disclosure

Generally, those skilled in the art will be able to easily produce nanoparticles used according to the disclosure. More specifically, the following elements will be noted:

For nanoparticles of core-shell type, based on a core of lanthanide oxide or oxyhydroxide, use may be made of a production process using an alcohol as solvent, as described for example in P. Perriat et al., J. Coll. Int. Sci, 2004, 273, 191; O. Tillement et al., J. Am. Chem. Soc., 2007, 129, 5076 and P. Perriat et al., J. Phys. Chem. C, 2009, 113, 4038.

For the POS matrix, several techniques can be used, derived from those initiated by Stoeber (Stoeber, W; J. Colloid Interf Sci 1968, 26, 62). Use may also be made of the process used for coating as described in Louis et al. (Louis et al., 2005, Chemistry of Materials, 17, 1673-1682) or international application WO 2005/088314.

In practice, synthesis of ultrafine nanoparticles is for example described in Mignot et al. Chem. Eur. J. 2013, 19, 6122-6136: Typically, a precursor nanoparticle of core/shell type is formed with a lanthanide oxide core (via the modified polyol route) and a polysiloxane shell (via sol/gel); this object has, for example, a hydrodynamic diameter of around 5-10 nm. A lanthanide oxide core of very small size (adjustable less than 10 nm) can thus be produced in an alcohol by means of one of the processes described in the following publications: P. Perriat et al., J. Coll. Int. Sci, 2004, 273, 191; O. Tillement et al., J. Am. Chem. Soc., 2007, 129, 5076 and P. Perriat et al., J. Phys. Chem. C, 2009, 113, 4038.

These cores can be coated with a layer of polysiloxane according to, for example, a protocol described in the following publications: C. Louis et al., Chem. Mat., 2005, 17, 1673 and O. Tillement et al., J. Am. Chem. Soc., 2007, 129, 5076.

Chelating agents specific for the intended metal cations (for example DOTAGA for Gd³⁺) are grafted to the surface of the polysiloxane; it is also possible to insert a part thereof inside the layer, but the control of the formation of the polysiloxane is complex and simple external grafting gives, at these very small sizes, a sufficient proportion of grafting.

The nanoparticles may be separated from the synthesis residues by means of a method of dialysis or of tangential filtration, for example on a membrane comprising pores of appropriate size.

The core is destroyed by dissolution (for example by modifying the pH or by introducing complexing molecules into the solution). This destruction of the core then allows a diffusion and a rearrangement of the polysiloxane layer (according to a mechanism of slow corrosion or collapse), which makes it possible to finally obtain a polysiloxane object with a complex morphology, the characteristic dimensions of which are of the order of magnitude of the thickness of the polysiloxane layer, i.e. much smaller than the objects produced up until now.

Removing the core thus makes it possible to decrease from a particle size of approximately 5-10 nm in diameter to a size below 8 nm, for example between 2-8 nm. The number of M for a nanoparticle size can be evaluated by virtue of the M/Si atomic ratio measured by EDX. Typically, this number of M per ultrafine nanoparticle may be comprised between 5 and 50.

In one specific embodiment, the nanoparticle according to the disclosure comprises a chelating agent which has an acid function, for example DOTA or DOTAGA. The acid function of the nanoparticle is activated for example using EDC/NHS (1-ethyl-3-(3-dimethylaminopropyl) carbodiimide/N-hydrosuccinimide) in the presence of an appropriate amount of targeting molecules. The nanoparticles thus grafted are then purified, for example by tangential filtration.

Alternatively, the nanoparticles according to the present disclosure may be obtained or obtainable by a synthesis method (“one-pot synthesis method”) comprising the mixing of at least one hydroxysilane or alkoxysilane which is negatively charged at physiological pH and at least one chelating agent chosen from polyamino polycarboxylic acids with
at least one hydroxysilane or alkoxysilane which is neutral at physiological pH, and/or
at least one hydroxysilane or alkoxysilane which is positively charged at physiological pH and comprises an amino function, wherein:
the molar ratio A of neutral silane(s) to negatively charged silane(s) is defined as follows: 0≤A≤6, preferably 0.5≤A≤2;
the molar ratio B of positively charged silane(s) to negatively charged silane(s) is defined as follows: 0≤B≤5, preferably 0.25≤B≤3;
the molar ratio C of neutral and positively charged silanes to negatively charged silane(s) is defined as follows 0<C≤8, preferably 1≤C≤4.

According to a more specific embodiment of such one pot synthesis method, the method comprises the mixing of at least one alkoxysilane which is negatively charged at physiological pH, said alkoxysilane being chosen among APTES-DOTAGA, TANED, CEST and mixtures thereof, with
at least alkoxysilane which is neutral at physiological pH, said alkoxysilane being chosen among TMOS, TEOS and mixtures thereof, and/or
APTES which is positively charged at physiological pH,
wherein:
the molar ratio A of neutral silane(s) to negatively charged silane(s) is defined as follows: 0≤A≤6, preferably 0.5≤A≤2;
the molar ratio B of positively charged silane(s) to negatively charged silane(s) is defined as follows: 0≤B≤5, preferably 0.25≤B≤3;
the molar ratio C of neutral and positively charged silanes to negatively charged silane(s) is defined as follows 0<C≤8, preferably 1≤C≤4.

According to a specific embodiment, the one pot synthesis method comprises the mixing of APTES-DOTAGA which is negatively charged at physiological pH with
at least one alkoxysilane which is neutral at physiological pH, said alkoxysilane being chosen among TMOS, TEOS and mixtures thereof, and/or
APTES which is positively charged at physiological pH,
wherein:
the molar ratio A of neutral silane(s) to negatively charged silane(s) is defined as follows: 0≤A≤6, preferably 0.5≤A≤2;
the molar ratio B of positively charged silane(s) to negatively charged silane(s) is defined as follows: 0≤B≤5, preferably 0.25≤B≤3;
the molar ratio C of neutral and positively charged silanes to negatively charged silane(s) is defined as follows 0<C≤8, preferably 1≤C≤4.

### AGuIX Nanoparticles

In a more particularly preferred embodiment, said gadolinium-chelated polysiloxane based nanoparticle is the ultrafine AGuIX nanoparticle of the formula below:

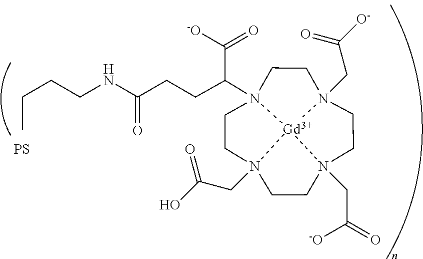

wherein PS is polysiloxane and n is, on average, about 10, and having a hydrodynamic diameter of 5±2 nm and a mass of between 2 and 200 kDa, preferably between 5 and 30 kDa, even more preferably 8 and 12 kDa, for example about 10±1 kDa.

Said AguIX nanoparticle can also be described by the average chemical formula:

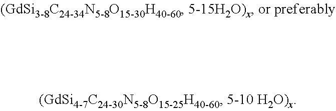

In the present disclosure, the terms AGuIX and AGuIX® may be used interchangeably.

### Pharmaceutical Formulations of the Nanoparticles for Use According to the Disclosed Methods

When employed as pharmaceuticals, the compositions comprising said high-Z nanoparticles for use as provided herein can be administered in the form of pharmaceutical formulation of a suspension of nanoparticles. These formulations can be prepared as described herein or elsewhere, and can be administered by a variety of routes, depending upon whether local or systemic treatment is desired and upon the area to be treated.

In a particular embodiment, said pharmaceutical formulations for use as described herein, contain, as the active ingredient, a suspension of high-Z containing nanoparticles, as provided herein, in combination with one or more pharmaceutically acceptable carriers (excipients). In making a pharmaceutical formulation provided herein, the nanoparticle composition may be, for example, mixed with an excipient or diluted by an excipient. When the excipient serves as a diluent, it can be a solid, semi-solid, or liquid material, which acts as a vehicle, carrier, or medium for the nanoparticle composition.

Thus, the pharmaceutical formulations can be in the form of powders, lozenges, elixirs, suspensions, emulsions, solutions, syrups, aerosols (as a solid or in a liquid medium), sterile injectable solutions, sterile packaged powders, and the like.

In specific embodiments, said pharmaceutical formulation for use as described herein, is sterile lyophilized powder, contained in a pre-filled vial to be reconstituted, for example in an aqueous solution for intravenous injection. In specific embodiments, said lyophilized powder comprises, as the active ingredient, an efficient amount of said high-Z containing nanoparticles, typically gadolinium-chelated polysiloxane based nanoparticles, and more specifically AguIX nanoparticles as described herein. In certain specific embodiments, said lyophilized powder contains either about between 200 mg and 15 g per vial, for example between 280 and 320 mg of AguIX per vial, typically 300 mg of AguIX per vial or about between 800 mg and 1200 mg, for example 1 g of AguIX per vial.

Such powder may further contain one or more additional excipients, and in particular CaCl₂), for example between 2 and 5 mg of CaCl₂), typically 4.4 mg of CaCl₂) per g of AguIX.

Said lyophilized powder may be reconstituted in an aqueous solution, typically water for injection. Accordingly, in specific embodiments, said pharmaceutical solution for use according to the present disclosure is a solution for injection, comprising, as the active ingredient, an efficient amount of said high-Z containing nanoparticles, typically gadolinium-chelated polysiloxane based nanoparticles, and more specifically AguIX nanoparticles as described herein. For example, said solution for injection for use in the methods as disclosed herein is a solution of gadolinium-chelated polysiloxane based nanoparticles, typically AguIX nanoparticles, between 50 and 150 mg/mL, for example 80 and 120 mg/mL, typically 100 mg/mL, optionally comprising one or more additional pharmaceutically acceptable excipient, for example between 0.2 and 0.6 mg/ml of CaCl₂), typically 0.44 mg/ml of CaCl₂).

In specific embodiment, said pharmaceutical formulation for use as described herein, is an aqueous solution, contained in a vial, comprising, as the active ingredient, an efficient amount of said high-Z containing nanoparticles, typically gadolinium-chelated polysiloxane based nanoparticles, and more specifically AguIX nanoparticles as described herein. In certain specific embodiments, said vial contains about between 1 and 5 g of AguIX, typically about between 2 g and 3 g of AguIX, for example 2.5 g of AguIX. In certain embodiments, the aqueous solution can be used to prepare a solution for injection as mentioned above, or is a solution for injection as mentioned above.

### Methods of Treatment of the Present Disclosure

The present disclosure relates to high-Z element containing nanoparticles for use in a method of treating a tumor by radiopharmaceutical therapy, in a subject in need thereof, the method comprising a combined administration of an efficient amount high-Z element containing nanoparticles and of an efficient amount of radionuclide containing therapeutic radiopharmaceutical. As used herein, the terms “high-Z element containing nanoparticles” refer to the nanoparticles described in the previous sections.

The present disclosure also relates to a method of treating a tumor in a subject in need thereof by radiopharmaceutical therapy, comprising co-administering to the subject, in particular according to the administration regimen disclosed herein, an effective amount high-Z element containing nanoparticles and an efficient amount of a radionuclide containing therapeutic radiopharmaceutical as herein disclosed.

The present disclosure also relates to the use of high-Z element containing nanoparticles and of a radionuclide containing therapeutic radiopharmaceutical as herein disclosed in the preparation of a medicament for the treatment of a tumor in a subject in need thereof by radiopharmaceutical therapy, wherein an efficient amount of the high-Z element containing nanoparticles and an efficient amount of the therapeutic radiopharmaceutical are to be co-administered, in particular according to the administration regimen disclosed herein.

According to the present disclosure, the term “radiopharmaceutical therapy”, also referred as “targeted radionuclide therapy” (abbreviated TRT), “targeted radiopharmaceutical therapy” or “molecular radiotherapy” or “radioimmunotherapy” or “targeted radiotherapy” refers to the treatment of diseases of oncological nature with a radionuclide containing therapeutic radiopharmaceutical. In radiopharmaceutical therapy, radiation is systemically or locally delivered to the tumor using a therapeutic radiopharmaceutical comprising a radionuclide able to deliver ionizing radiation directly to tumor cells. Ionizing radiation deposits energy that injures or destroys cells in the area being treated (the target tissue and the so called “targeted effects”) by damaging their genetic material, making it impossible for these cells to continue to grow. Typically, said ionizing radiations are alpha, beta particles and Auger electrons. Radiation can also damage non-irradiated cells at short (Bystander effects) or long distance (systemic or immune effects) of the irradiated cells through intercellular communications.

According to the present disclosure, the term “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); and (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease or reducing or alleviating one or more symptoms of the disease. In particular, with reference to the treatment of a tumor, the term “treatment” may refer to the inhibition of the growth of the tumor, the reduction of the size of the tumor, or the total destruction of the tumor.

According to the present disclosure, the terms “efficient amount” or “therapeutically efficient amount” of an active principle ingredient (for example a therapeutic radiopharmaceutical) refer to an amount of the active principle ingredient that will elicit the biological or medical response of a subject, for example, ameliorate the symptoms, alleviate conditions, slow or delay disease progression, or prevent a disease, either alone or in combination with another active principle ingredient (e.g. in combination with a high-Z element containing nanoparticle).

According to the present disclosure, the efficient amount of therapeutic radiopharmaceutical refers to an amount of therapeutic radiopharmaceutical administered to a patient to treat a tumor in a context of radiopharmaceutical therapy, e.g. by inducing tumor regression or elimination by destroying tumor's structure and/or killing tumor cells. Such efficient amount therapeutic radiopharmaceutical is generally expressed in terms of activity (Mega Becquerel, MBq or Giga Becquerel, GBq) and may also referred to as “total injected activity”. Such efficient amount is generally designed to deliver a suitable radiation dose (in Gray, Gy) to the tumor.

According to the present disclosure, the efficient amount of therapeutic radiopharmaceutical may be administered in a fractionated dose regimen, i.e. a regimen wherein a total dose of therapeutic radiopharmaceutical (i.e. a total injected activity) is divided into several, smaller doses each herein referred to as “therapeutic radiopharmaceutical fractionated dose” administered to the patient in multiple administration cycles, e.g. over a period of several days or months. Alternatively, the therapeutic radiopharmaceutical may be administered in one single dose. The efficient amount of therapeutic radiopharmaceutical depends on several factors such as the administration regimen, the therapeutic radiopharmaceutical, the patient weight, the location and severity of the tumor.

In an embodiment, the efficient amount of therapeutic radiopharmaceutical administered to the subject is comprised between 0.5 MBq and 100 GBq, preferably between 10 MBq and 50 GBq.

In an embodiment, the therapeutic radiopharmaceutical is administered in a fractionated regimen having 4 to 20 administration cycles, each fractionated dose having an activity comprised between 0.5.MBq and 100 GBq, preferably 1 Mbq and 10 Gbq, more preferably between 2 MBq and 8 GBq.

In a particular embodiment, the therapeutic radiopharmaceutical is ¹⁷⁷Lu-DOTATATE, and the efficient amount of therapeutic radiopharmaceutical is administered in a fractionated regimen having 2 to 6 administration cycles, preferably 4 administration cycles, each fractionated dose having an activity comprised between 1 and 10 GBq, preferably between 5 GBq and 10 GBq, more preferably between 7 and 9 GBq, for example of 7.4 GBq. For example, ¹⁷⁷Lu-DOTATATE may be administered in a fractionated regime having 4 administration cycles, each dose having an activity comprised between 7 and 8 GBq, typically of 7.4 GBq.

In an embodiment, the efficient amount of therapeutic radiopharmaceutical is designed to deliver to the tumor a radiation dose (in Gray, Gy) of at least 50 mGy, preferably of at least 500 mGy, more preferably of at least 1 Gy to the tumor. It should be noted that radionuclide imaging techniques result in an exposure of less than 50 mGy whereas Gy is achieved for therapy using radionuclides.

According to the present disclosure, the efficient amount of nanoparticles refers to an amount of nanoparticle that allows to enhance the therapeutic efficacy of the therapeutic amount of therapeutic radiopharmaceutical administered to a patient to treat a tumor. The efficient amount of nanoparticles may depend on several factors such as the type, effective amount and the administration regimen of therapeutic radiopharmaceutical administered to the patient.

According to the present disclosure, the efficient amount of nanoparticles is preferably administered in a fractionated dose regimen, i.e. for each dose of therapeutic radiopharmaceutical administered to the patient, a total dose of nanoparticles is divided into several, smaller nanoparticles doses each herein referred to as a “nanoparticles fractionated dose” and administered in multiple administration cycles to the patient, e.g. over a period of 4 h to 72 h before or after the administration of each dose of therapeutic radiopharmaceutical.

According to the present disclosure, the terms “patient” and “subject” which are used herein interchangeably refer to any member of the animal kingdom, including mammals and invertebrates. For example, mice, rats, other rodents, rabbits, dogs, cats, swine, cattle, sheep, horses, primates, fish, and humans. Preferably, the subject is a mammal, more preferably a human, including for example a subject that has a tumor.

According to the present disclosure, the terms “combined administration”, “co-administration”, or “concomitant administration” refers to a combined administration of at least two therapeutic agents, where a first agent, typically a therapeutic radiopharmaceutical compound is administered at the same time or separately within time intervals, with a second agent, typically, a high-Z element containing nanoparticle (preferably AguIX) in the same subject in need thereof, in particular according to the administration regimen disclosed herein, where these time intervals allow that the combined partners show a cooperative or synergistic effect for treating a tumor. It is not intended to imply that the therapeutic agents must be administered at the same time and/or formulated for delivery together although these methods of delivery are within the scope described herein. As used herein, the terms “combined administration”, “co-administration”, and “concomitant administration” are opposed to monotherapy which involves the administration of a single therapeutic agent. The therapeutic radiopharmaceutical can be administered concurrently with or prior to, or subsequent to one or more other additional therapies or therapeutic agents. The terms are also meant to encompass treatment regimens in which at least one or both of the agents are administered in a fractionated regimen. The terms are also meant to encompass treatment regimens in which the agents are not necessarily administered by the same route of administration.

In an embodiment, the combined administration for use according to the invention induces oxidative cell death in the tumor cells.

In an embodiment, the combined administration for use according to the invention decreases the antioxidant capacity of the tumor cells, and/or increases the accumulation of reactive oxygen species (ROS) in the tumors cells, resulting in oxidative cell death of the tumor cells.

### Therapeutic Radiopharmaceutical

According to the present disclosure, the radiopharmaceutical therapy may be carried out using any kind of therapeutic radiopharmaceutical.

In the present disclosure, the terms “radionuclide containing therapeutic radiopharmaceutical”, “therapeutic radiopharmaceutical” and “targeted radiopharmaceutical”, “radiopharmaceutical compound” or “radiopharmaceutical” which are used herein interchangeably refer to a pharmaceutical compound intended to be administered to the subject in need thereof, the pharmaceutical compound comprising a radionuclide able to emit ionizing radiation, in particular to deliver ionizing radiation directly and specifically to tumor cells, to the microenvironment of tumor cells and/or to an organ harboring tumor cells.

According to the present disclosure, the term “radionuclide” refers to any radioactive isotope of an element emitting one or more ionizing radiation selected from beta minus particles, alpha minus particles, Auger electrons.

In an embodiment the radionuclide is selected from ¹⁷⁷Lu, ¹⁶¹Tb, ¹⁸⁶Re, ¹³¹I, ⁹⁰Y, ²²⁵Ac/²¹³Bi, ²²³Ra, ²¹²Pb/²¹²Bi, ²²⁷Th, ²¹¹At, ⁹⁷Ru, ¹⁰³Pd, ⁶⁷Ga, ^{195m}Pt, ^{193m}Pt, ¹²⁵I, ¹¹¹In, and mixtures thereof.

In an embodiment, the radionuclide is a beta particle emitter selected from ¹⁷⁷Lu, ¹⁶¹Tb, ¹⁸⁶Re, ¹³¹I, ⁹⁰Y and mixtures thereof.

In an embodiment, the radionuclide is an alpha particle emitter selected from ²²⁵Ac/²¹³Bi, ²²³Ra, ²¹²Pb/²¹²Bi, ²²⁷Th, ²¹¹At and mixtures thereof.

In an embodiment, the radionuclide is an Auger electrons emitter selected from ⁹⁷Ru, ¹⁰³Pd, ⁶⁷Ga, ^{195m}Pt, ^{193m}Pt, ¹²⁵I, ¹¹¹In and mixtures thereof.

Some of the radionuclides described herein such as ¹⁷⁷Lu, ¹³¹I, ¹¹¹In emit gamma/X rays or beta plus particles in addition to the ionizing radiation mentioned above and can thus be monitored by SPECT imagery (Single Photon Emission Computed Tomography).

The targeting of the radionuclide to the tumor may be provided by a cancer-targeting moiety or be intrinsic to the radionuclide, as detailed below.

In the present disclosure, the radionuclide containing therapeutic radiopharmaceutical is distinct from the high Z element containing nanoparticle.

### Radionuclides Linked to a Cancer-Targeting Moiety

In an embodiment, the radionuclide is linked to a cancer-targeting moiety.

According to the present disclosure, the term “linked” means that the cancer-targeting moiety and the radionuclide are chemically linked by a covalent link, optionally trough a linker moiety, or that the cancer targeting moiety comprises a chelating moiety and that the radionuclide is complexed to a chelating moiety of the cancer targeting moiety.

In an embodiment, the therapeutic radiopharmaceutical is of formula N—(X—)M, wherein N is the radionuclide, M is the cancer-targeting moiety, and X is an optional chelating moiety (Ch) or linker moiety (L). In an embodiment, the radionuclide N is complexed to the cancer-targeting moiety through the chelating moiety (Ch). In another embodiment, the radionuclide is covalently linked to the cancer-targeting moiety M through the linker moiety (L).

According to the present disclosure, the term “cancer-targeting moiety” refers to a moiety providing a targeting for tumor cells.

In an embodiment, the cancer targeting moiety exhibits a capacity to recognize and bind to one or more sites or antigens, for example a cell surface receptor, specific of tumor cells, of the tumor microenvironment or of the organ harboring the tumor cells.

The cancer-targeting moiety may be an antibody of a binding fragment thereof, a peptide or a small-molecule ligand that preferably binds specifically to a tumor antigen or tumor-associated antigen.

According to the present disclosure, “specifically binds to” or “binds specifically to” or “targets” means that the cancer-targeting moiety (e.g. the antibody) binds to the stated antigen with greater affinity than it binds unrelated antigens. Preferably such affinity is at least 10-fold greater, more preferably at least 100-fold greater, and most preferably at least 1000-fold greater than the affinity of the cancer-targeting moiety for unrelated antigens. In a particular embodiment, “specifically binds” means that the cancer-targeting moiety binds to the stated target and not to other antigens.

As used herein, the term “tumor antigen” or “cancer antigen” refers to any protein produced in a tumor cell that has an abnormal sequence or structure due to mutation can act as a tumor antigen. Mutation of protooncogenes and tumor suppressors which lead to abnormal protein production are the cause of the tumor and thus such abnormal proteins are called tumor-specific antigens. Examples of tumor antigens include the abnormal products of ras and p53 genes. “Tumor antigen” also refers to “Tumor-associated antigens” which are proteins with a mutation of other genes unrelated to the tumor formation which may lead to synthesis of abnormal proteins. The term also encompasses other cellular antigens, which can be native, but may be targeted by anti-cancer drugs to eliminate the cells expressing such antigens.

In a particular embodiment, the cancer-targeting moiety is an antibody which binds specifically to a tumor antigen or tumor-associated antigen.

According to the present disclosure, the term “antibody” refers to any whole antibody molecule e.g., of any isotype (IgG, IgA, IgM, IgE, etc), containing an immunoglobulin binding domain that specifically binds an antigen such as a tumor antigen or tumor-associated antigen. The term antibody includes polyclonal, monoclonal, or other purified preparations of antibodies and recombinant antibodies. The term “antibody binding fragment” refers to fragments of whole antibodies that retain the property to specifically bind an antigen. Antibodies can be fragmented using conventional techniques and the fragments screened for their interaction with an antigen of interest. Thus, the term “fragment” includes segments of proteolytically-cleaved or recombinantly-prepared portions of an antibody molecule that are capable of binding specifically to a certain antigen. Non-limiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites.

In an embodiment the cancer-targeting moiety specifically binds to one or more cancer cells antigen selected from HER2 (human epidermal growth factor receptor 2), EGFR (Epithelial Growth Factor Receptor), GRBR (gastrin-releasing peptide receptor), CD20, CD33, CD37, somatostatin receptors, prostate-specific membrane antigen (PSMA), AMHRII (anti-Mullerian hormone receptor type 2), and TYRP1/gp75 (Tyrosinase related protein 1).

In an embodiment the cancer-targeting moiety specifically binds at least one antigen of the tumor microenvironment, for example a cancer-associated fibroblasts (CAFs) antigen such as fibroblast activation protein a (FAP), or an immune checkpoint antigen such as cytotoxic T-lymphocyte-associated protein 4 (CTLA-4), programmed death 1 receptor (PD-1) or its ligand PD-L1.

In an embodiment, the cancer-targeting moiety is an anti-HER2 antibody such as trastuzumab, pertuzumab, or ibritumomab (also referred to as ibritumomab tiuxetan, commercialized under the trademark Zevalin®), an anti-EGFR antibody such as cetuximab or panitumumab, an anti-CD20 antibody such as rituximab or zevalin, an anti-CD33 antibody such as lintuzumab, an anti-CD37 antibody such as Otlertuzumab (TRU-016), mAB 37.1 (BI 836826) or IMGN529 (K7153A-DM1), an anti-AMHRII antibody such as murlentamab, or an anti-TYRP1/gp75 antibody such as IMC-20D7S.

In an embodiment, the cancer-targeting moiety is a somatostatin analog such as Octreotide (DOTATOC) or Octreotate (DOTATATE) which targets SSTR2 receptors.

In an embodiment the cancer-targeting moiety is a PSMA small-molecule ligand such as 617 ligand, I&T ligand, R2 ligand or MIP-1095 ligand.

In an embodiment, the therapeutic radiopharmaceutical is selected from a ¹⁷⁷Lu-anti-HER2 antibody such as ¹⁷⁷Lu-trastuzumab, a ¹⁷⁷Lu-somatostatin analog which targets SSTR2 receptors such as ¹⁷⁷Lu-dotatate, a ¹⁷⁷Lu-PSMA ligand, an ⁹⁰Y-anti-CD20 antibody such as ⁹⁰Y-rituximab or ⁹⁰Y-ibritumomab, an ²¹²Pb-anti-HER2 antibody, and an ¹⁷⁷Lu-anti CD37 antibody.

Radionuclides with Intrinsic Targeting

In another embodiment, the therapeutic radiopharmaceutical consists of a radionuclide with intrinsic targeting, i.e. a radionuclide having an intrinsic affinity for tumor cells, the microenvironment of tumor cells, or for an organ harboring tumor cells. In that embodiment, the therapeutic radiopharmaceutical does not possess a cancer-targeting moiety as described above.

Such therapeutic radiopharmaceutical may be selected from lodine 131 (¹³¹I) or Radium 223 (²²³Ra).

Iodine 131 naturally accumulates in the thyroid gland and can be used as a therapeutic radiopharmaceutical to treat thyroid tumors.

Radium 223 naturally accumulates in the bone and can be used as a therapeutic radiopharmaceutical to treat bone metastases from prostate or breast cancer.

### Nanoparticles

In an embodiment, the radionuclide containing therapeutic radiopharmaceutical and the nanoparticles do not have the same targeting characteristics.

In an embodiment, the nanoparticles are not functionalized with molecules which allow targeting of the nanoparticles to specific tissues, in particular to tumors.

In an embodiment, the nanoparticles are not linked to a cancer-targeting moiety as described herein.

In an embodiment, the nanoparticles reach the tumor, in particular the tumor cells, only due to passive targeting. In an embodiment, the nanoparticles colocalize sufficiently with the radionuclide containing therapeutic radiopharmaceutical so that the nanoparticles have a radiosensitizing effect on the radiopharmaceutical, preferably specifically at the tumor.

In an embodiment, the nanoparticles have a radiosensitizing effect on the radionuclide containing therapeutic radiopharmaceutical, preferably specifically at the tumor.

In an embodiment, the nanoparticles have a radiosensitizing effect on the lysosomes of the tumor cells.

In an embodiment, the radiosensitizing effect is sufficient to generate one or more of the following in the tumor cells: (i) enhanced lysosomal disruption in the tumor cells, (ii) enhanced release of iron in the tumor cells, (iii) enhanced ROS production in the tumor cells (iv) enhanced lipid peroxidation in the tumor cells, and/or (v) oxidative cell death of the tumor cells.

In an embodiment, the radiosensitizing effect is mediated at least in part by ferroptosis.

In an embodiment, the radiosensitizing effect is sufficient to induce oxidative cell death in a tumor cell.

In an embodiment, the nanoparticles are AGuix nanoparticles as described in the previous sections.

### Administration Regimen

The present disclosure relates to high-Z element containing nanoparticles for use in a method of treating a tumor by radiopharmaceutical therapy, in a subject in need thereof, the method comprising the combined administration of high-Z element containing nanoparticles and of a radionuclide containing therapeutic radiopharmaceutical.

The inventors have shown that such combined administration, in particular when the nanoparticles are administered to the subject in a fractionated dose regimen, increases tumor regression when compared with administration of radionuclide containing therapeutic radiopharmaceutical alone. It is believed that the advantageous effect of the combined administration according to the method of treatments of the present disclosure are as follows:
(i) to enhance the therapeutic efficacy of an amount of a therapeutic radiopharmaceutical administered to a patient to treat a tumor,
(ii) to reduce the efficient amount of therapeutic radiopharmaceutical that needs to be administered to a patient to treat a tumor, thereby allowing to reduce radiation-induced toxicity for the patient.

In an embodiment, the high-Z element containing nanoparticles and the therapeutic radiopharmaceutical are administered simultaneously, separately, or sequentially. In a particular embodiment, the therapeutic radiopharmaceutical is administered prior to or subsequent to the nanoparticles.

In an embodiment, the nanoparticles are administered to the subject in a fractionated dose regimen.

In an embodiment, the therapeutic radiopharmaceutical is administered to the subject in a fractionated dose regimen.

In an embodiment, the nanoparticles and the therapeutic radiopharmaceutical are both administered to the subject in a fractionated dose regimen, i.e. the therapeutic radiopharmaceutical is administered to the subject in a fractionated dose regimen, and for each dose of therapeutic radiopharmaceutical, the nanoparticles are administered in a fractionated dose regimen.

In an embodiment, the nanoparticles fractionated dose regimen is designed to potentiate the therapeutic activity of a dose of therapeutic radiopharmaceutical at least during the efficient half-life of the said therapeutic radiopharmaceutical dose. In the present disclosure, the efficient half-life (T_{eff}) of a therapeutic radiopharmaceutical dose may be calculated taking into account the radioactive decay of the radionuclide (T_{Phys}) and the biological half-life (T_{biol}) of the radiopharmaceutical using the formula 1/T_{eff}=(1/T_{phys})+(1/T_{biol}). In french, the term efficient half-life (Terr) is said “période effective”.

In an embodiment, the fractionated dose regimen of nanoparticles comprises 2 to 10 fractionated doses of nanoparticles for each dose of therapeutic radiopharmaceutical administered to the subject.

In an embodiment, the nanoparticles administration is fractionated between 24 h and 72 h post-administration of a dose of therapeutic radiopharmaceutical.

In an embodiment, the fractionated dose regimen of nanoparticles is administered on day 1, 2, 3, 4 and/or 5 post-administration of a dose of therapeutic radiopharmaceutical.

In an embodiment, the fractionated dose regimen of nanoparticles is administered on day 1, 2, 3, 4 and/or 5 after administration of each dose of therapeutic radiopharmaceutical.

In a preferred embodiment, the fractionated dose regimen of nanoparticles is administered once daily or once every two days.

The first fractioned dose of nanoparticles may be administered before or after the first administration of therapeutic radiopharmaceutical to the subject in need thereof.

In an embodiment, the first fractioned dose of nanoparticles is administered 5 minutes, 15 minutes, 30 minutes, 45 minutes, 1 hour, 2 hours, 4 hours, 6 hours, 12 hours, 24 hours, 48 hours or 72 hours, preferably between 24 h and 72 h, post-administration of a dose of therapeutic radiopharmaceutical.

In an embodiment, the first fractioned dose of nanoparticles is administered 5 minutes, 15 minutes, 30 minutes, 45 minutes, 1 hour, 2 hours, 4 hours, 6 hours, 12 hours, 24 hours, 48 hours or 72 hours, preferably between 24 h and 72 h, after the first administration of the dose of therapeutic radiopharmaceutical to the subject in need thereof.

In an embodiment, the fractionated doses of nanoparticles are administered with a time lapse between two fractionated doses comprised between 4 h and 48 h, preferably between 4 h and 12 h, more preferably between 4 h and 10 h, for example with a time lapse of 6 hours.

In an embodiment, the nanoparticle fractionated regimen is designed depending on:
(i) the tumor elimination and/or renal retention of the nanoparticles, to avoid too high toxicity, and
(ii) the time the therapeutic radiopharmaceutical is retained at the tumor site.

Without wishing to be bound by any theory, the inventors believe that when the therapeutic radiopharmaceutical is a radionuclide linked to an antibody, a dose of therapeutic radiopharmaceutical is fixed to the tumor from 24 h to 168 h post-administration to the patient, with a maximum between 24 h and 72 h. In that event, the inventors believe that the nanoparticles administration should preferably be fractionated between 24 h and 72 h post-administration of a dose of therapeutic radiopharmaceutical to maximize the potential of the dose of therapeutic radiopharmaceutical.

### Patient Selection

In an embodiment, the present disclosure relates to a method of treating a tumor by radiopharmaceutical therapy, in a subject in need thereof, the method comprising administering the high-Z element containing nanoparticles in combination with a dose of therapeutic radiopharmaceutical, wherein the nanoparticles allow to reduce the dose of therapeutic radiopharmaceutical that needs to be administered to the subject in need thereof, when compared to the dose to be administered to a subject treated only by radiopharmaceutical therapy.

In an embodiment, the method of the present disclosure is applied to subjects which cannot receive the standard effective dose of a targeted radionuclide treatment.

In a preferred embodiment, the method of the present disclosure is applied to subjects having radioresistant tumors. The radioresistant tumors include but are not limited to renal tumor, melanoma, thyroid tumors, colorectal tumors.

In an embodiment, the therapeutic activity of said therapeutic radiopharmaceutical is due to both the biological action of the cancer-targeting moiety and the effects of ionizing radiation of the radionuclide.

### The Tumor to be Treated

According to the present disclosure, the term “tumor” refers to an abnormal mass of tissue in which the growth of the mass exceeds the growth of normal tissue and is not as coordinated as the growth of normal tissue. A tumor may be “benign” or “malignant,” depending on the following characteristics: degree of cell differentiation (including morphology and function), growth rate, local invasion and metastasis. “benign tumors” are generally well differentiated, have significantly slower growth than malignant tumors, and remain localized to the site of origin. In addition, benign tumors do not have the ability to infiltrate, invade, or metastasize to distant locations. In some cases, some “benign” tumors may later develop into malignant tumors, which may be due to additional genetic changes in the tumor cell subpopulation of the tumor, and these tumors are referred to as “pre-cancerous tumors”. An exemplary pre-cancerous tumor is a teratoma. In contrast, “malignant tumors” are generally poorly differentiated (anaplasia) and have significantly rapid growth with progressive infiltration, invasion and destruction of surrounding tissue. In addition, malignant tumors often have the ability to metastasize to distant locations. The terms “malignant tumor” and “cancer” are used herein interchangeably.

In specific embodiments, the tumor to be treated express tumor antigens which are specifically targeted by the radiopharmaceutical compound for use in the methods of the disclosure.

Exemplary cancers include, but are not limited to, acoustic neuroma; adenocarcinoma; adrenal cancer; anal cancer; angiosarcoma (angiosarcoma) (e.g., lymphangioangiosarcoma, lymphangioendotheliosarcoma, angiosarcoma);

appendiceal carcinoma; benign monoclonal propionibacteria; biliary tract cancer (e.g., cholangiocarcinoma); bladder cancer; breast cancer (e.g., breast adenocarcinoma, breast papillary carcinoma, breast cancer, breast medullary carcinoma); brain cancer (e.g., meningioma, glioblastoma, glioma (e.g., astrocytoma, oligodendroglioma), medulloblastoma); bronchial cancer; carcinoid tumors; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial cancer; ependymoma; endothelial sarcoma (e.g., kaposi's sarcoma, multiple idiopathic hemorrhagic sarcoma); endometrial cancer (e.g., uterine cancer, uterine sarcoma); esophageal cancer (e.g., esophageal adenocarcinoma, barrett's adenocarcinoma); ewing's sarcoma; eye cancer (e.g., intraocular melanoma, retinoblastoma); familial hypereosinophilia; gallbladder cancer; stomach cancer (e.g., gastric adenocarcinoma); gastrointestinal stromal tumors (GIST); germ cell cancer; head and neck cancer (e.g., head and neck squamous cell carcinoma, oral cancer (e.g., oral squamous cell carcinoma), laryngeal cancer (e.g., laryngeal carcinoma, pharyngeal cancer, nasopharyngeal cancer, oropharyngeal cancer)); hematopoietic cancers (e.g., leukemias, such as Acute Lymphocytic Leukemia (ALL) (e.g., B-cell ALL, T-cell ALL), Acute Myelogenous Leukemia (AML) (e.g., B-cell AML, T-cell AML), Chronic Myelogenous Leukemia (CML) (e.g., B-cell CML, T-cell CML), and Chronic Lymphocytic Leukemia (CLL) (e.g., B-cell CLL, T-cell CLL)); lymphomas such as Hodgkin's Lymphoma (HL) (e.g., B-cell HL, T-cell HL) and non-Hodgkin's lymphoma (NHL) (e.g., B-cell NHL such as Diffuse Large Cell Lymphoma (DLCL) (e.g., diffuse large B-cell lymphoma), follicular lymphoma, chronic lymphocytic leukemia/small lymphocytic lymphoma (CLL/SLL), Mantle Cell Lymphoma (MCL), marginal zone B-cell lymphoma (e.g., mucosa-associated lymphoid tissue (MALT) lymphoma, lymph node marginal zone B-cell lymphoma, splenic marginal zone B-cell lymphoma), primary mediastinal B-cell lymphoma, Burkitt's lymphoma, lymphoplasmacytic lymphoma (i.e., Waldenstrom's macroglobulinemia), Hairy Cell Leukemia (HCL), immunoblastic large cell lymphoma, precursor B-lymphoblastic lymphoma and primary Central Nervous System (CNS) lymphoma, and T-cell NHL, such as precursor T lymphoblastic lymphoma/leukemia, Peripheral T Cell Lymphoma (PTCL) (e.g., Cutaneous T Cell Lymphoma (CTCL) (e.g., mycosis fungoides, sezary syndrome), angioimmunoblastic T cell lymphoma, extranodal natural killer T cell lymphoma, enteropathy-type T cell lymphoma, subcutaneous panniculitis-like T cell lymphoma, and anaplastic large cell lymphoma); a mixed state of one or more of the above leukemias/lymphomas; and Multiple Myeloma (MM)), heavy chain disorders (e.g., alpha chain disorders, gamma chain disorders, mu chain disorders); hemangioblastoma; hypopharyngeal carcinoma; inflammatory myofibroblast tumors; immune cell amyloidosis; kidney cancer (e.g., nephroblastoma, also known as wilms tumor, renal cell carcinoma); liver cancer (e.g., hepatocellular carcinoma (HCC), malignant hepatoma); lung cancer (e.g., bronchogenic carcinoma, Small Cell Lung Cancer (SCLC), non-small cell lung cancer (NSCLC), lung adenocarcinoma); leiomyosarcoma (LMS); mastocytosis (e.g., systemic mastocytosis); muscle cancer; myelodysplastic syndrome (MDS); mesothelioma; myeloproliferative disorders (MPDs) (e.g., Polycythemia Vera (PV), Essential Thrombocythemia (ET), Agnogenic Myeloid Metaplasia (AMM), also known as Myelofibrosis (MF), chronic idiopathic myelofibrosis, Chronic Myelogenous Leukemia (CML), Chronic Neutrophilic Leukemia (CNL), hypereosinophilic syndrome (HES)); neuroblastoma; neurofibromatosis (e.g., Neurofibromatosis (NF) type 1 or type 2, schwannomatosis (schwannomatosis)); neuroendocrine tumors (e.g., gastroenteropancreatic neuroendocrine tumor (GEP-NET), carcinoid tumors); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma (pancreatic andenocicepma), Intraductal Papillary Mucinous Neoplasm (IPMN), pancreatic islet cell carcinoma); penile cancer (e.g., paget's disease of the penis and scrotum); peritoneal cancer (e.g. primary peritoneal cancer or peritoneal carcinomatosis, i.e. a secondary peritoneal cancer that spreads to the abdominal cavity after developing elsewhere in the body such as from the gastrointestinal tract, pancreas, melanoma, breast, lung, and ovary), pineal tumor; primitive Neuroectodermal Tumors (PNT); a plasmacytoma; paraneoplastic syndromes (paraneoplastic syndromes); intraepithelial tumors; prostate cancer (e.g., prostate adenocarcinoma); rectal cancer; rhabdomyosarcoma; salivary gland cancer; skin cancer (e.g., Squamous Cell Carcinoma (SCC), Keratoacanthoma (KA), melanoma, Basal Cell Carcinoma (BCC)); small bowel cancer (e.g., appendiceal cancer); soft tissue sarcomas (e.g., Malignant Fibrous Histiocytoma (MFH), liposarcoma, Malignant Peripheral Nerve Sheath Tumor (MPNST), chondrosarcoma, fibrosarcoma, myxosarcoma); sebaceous gland cancer; small bowel cancer; sweat gland cancer; a synovial tumor; testicular cancer (e.g., seminoma, testicular embryonal carcinoma); thyroid cancer (e.g., papillary carcinoma of the thyroid, Papillary Thyroid Carcinoma (PTC), medullary thyroid carcinoma); cancer of the urethra; vaginal cancer; and vulvar cancer (e.g., paget's disease of the vulva).

In an embodiment, the tumor to be treated is a metastatic tumor.

According to the present disclosure, the term “metastatic tumor” refers to a tumor formed of tumor cells that come from somewhere else in the body than where the metastatic tumor is located. The term metastatic refer to the spread or metastasis of cancer cells from a primary or original tumor to another organ or tissue. In the course of metastasis, tumor cells break away from an original (primary) tumor, travel through the blood or lymph system, and form a new tumor in other organs or tissues of the body. The new, metastatic tumor is the same type of cancer as the primary tumor. In the organ or tissue where the secondary (metastatic) tumor is located, there is a “secondary tumor” of the tissue type of the primary or original tumor and not of the organ or tissue where it is located. For example, prostate cancer that has metastasized to bone is referred to as metastatic prostate cancer and includes cancerous prostate cancer cells that grow in bone tissue. In certain embodiment, metastatic tumors may be diffuse tumors, i.e. widely spread, not localized or confined tumors.

In an embodiment, the tumor is a HER2-positive tumor, such as a HER2-positive breast, bladder, pancreatic, ovarian, or stomach tumor.

In a preferred embodiment, the tumor is selected from peritoneal tumors including primary peritoneal tumors and secondary peritoneal tumors, neuroendocrine tumors including gastroenteropancreatic neuroendocrine tumors and pheochromocytoma or paragangliomas (PPGLs), prostate tumor, neuroblastoma, meningioma, lymphoma, Merkel cell carcinoma, breast tumor, renal cell tumor, and salivary gland carcinoma.

In a particular embodiment, the secondary peritoneal cancer tumor is a peritoneal carcinomatosis tumor, i.e. tumor that has spread to the abdominal cavity after developing elsewhere in the body such as from the gastrointestinal tract, pancreas, melanoma, breast, lung, and ovary.

In an embodiment, the present disclosure relates to high-Z element containing nanoparticles for use in a method of treating a tumor by radiopharmaceutical therapy, wherein the tumor is a HER-2 positive tumor, more specifically a HER-2 breast tumor, in a subject in need thereof, the method comprising a combined administration of:
an efficient amount of said high-Z element containing nanoparticles as described herein, more specifically AGuIX nanoparticles as described herein, and of
an efficient amount of an ¹⁷⁷Lu-anti-HER2 antibody and more specifically ¹⁷⁷Lu-trastuzumab.

In an embodiment, the present disclosure relates to high-Z element containing nanoparticles for use in a method of treating a tumor by radiopharmaceutical therapy, wherein the tumor is a SSTR2 positive tumor such as SSTR2 midgut neuroendocrine tumor in a subject in need thereof, the method comprising a combined administration of:
an efficient amount of said high-Z element containing nanoparticles as described herein, and more specifically AGuIX nanoparticles as described herein, and of
an efficient amount of a somatostatin analog such as Octreotide (DOTATOC) or Octreotate (DOTATATE) which targets SSTR2 receptors, and more specifically ¹⁷⁷Lu-DOTATATE.

### Administration Route

In an embodiment, the nanoparticles and/or the therapeutic radiopharmaceutical are administered to the subject using routes selected from local (intra-tumoral (IT), intra-arterial (IA), subcutaneous, intravenous (IV), intradermic, airways (inhalation), intra-peritoneal, intramuscular, intra-thecal, intraocular or oral route.

### Brief Intraperitoneal Radioimmunotherapy (BIP-RIT) or Brief Intraperitoneal TRT

In an embodiment, the present disclosure relates to high-Z element containing nanoparticles for use in a method of treating a tumor by radiopharmaceutical therapy, in a subject in need thereof, the method comprising:
(i) a cytoreductive surgery of a tumor that have originated in or spread to the abdominal cavity, such as appendiceal tumor, colon tumor, gastric tumor, ovarian tumor, and peritoneal mesothelioma,
(ii) the combined administration of high-Z element containing nanoparticles and of a radionuclide containing therapeutic radiopharmaceutical according to the method as described herein, more specifically AGuIX nanoparticles as described herein, within the peritoneal cavity, and
(iii) optionally, the washing of the peritoneal cavity, for example with saline solution using a peristaltic pump, to remove the therapeutic radiopharmaceutical that is not bound to the tumor.

In the present disclosure, the term “cytoreductive surgery” also referred to as “CRS” means a surgical procedure that aims to reduce the amount of tumor cells in the abdominal cavity for patients with tumors that have spread intraabdominally (peritoneal carcinomatosis). It is often used to treat ovarian cancer but can also be used for other abdominal malignancies.

### EXAMPLES

The present disclosure is further illustrated by the following examples.

### Example 1

### Materials and Methods

### Cell Lines

The SK-OV-3-luc cell line, with a human ovarian serous cystadenocarcinoma origin was selected. Cells were obtained from the American Type Culture Collection (ATCC), and have been transfected to express the Luciferase gene, which allows us to follow intraperitoneal (IP) tumor growth by bioluminescence imaging. Cells were cultured in DMEM/F12 culture medium supplemented with 10% fetal calf serum and 1% penicillin/streptomycin in a 5% CO₂ atmosphere at 37° C. Hygromicine 0.1 mg/mL was added to the culture medium to select cells expressing the luciferase gene. SK-OV-3-luc express the EGFR family HER2 receptor, which can be targeted with Trastuzumab (Herceptin®, Roche). Besides, the cell line presents two major features of a HGSOC: resistance to platinum and p53 mutation. In vitro radiosensitization and AGuIX® cytotoxicity have been determined in additional cancer models: A431 (human vulvar epidermal carcinoma), B16F10 (murine melanoma) and MiaPaca2 (human pancreatic cancer, expressing somatostatin sst2), obtained from the American Type Culture Collection (ATCC).

### Animals

Athymic female Swiss nude mice (6-8 weeks old) (Charles River) were kept in the animal facility for 1 week before use. They were housed at 22° C. and 55% humidity, with a light-dark cycle of 12 h and ad libitum food and water. Body weight was monitored weekly, and mice were examined throughout the study. They were intraperitoneally (IP) xenografted with 3×10⁶ SK-OV-3-luc cells in 200 μl DMEM-F12 serum-free medium. Tumor growth follow-up was monitored by bioluminescence imaging. Mouse wellbeing was monitored throughout the study, and no clinical signs of pain or distress were seen. 4 weeks post-treatment, tumor nodules were recovered and measured. Results are presented as the mean tumor mass (mg) against the different treatment groups.

### Treatments

In vitro

Trastuzumab (Herceptin, Roche) conjugated with p-SCN-benzyl-DOTA (Macrocyclics, Plano, Tx, USA) were labeled with ¹⁷⁷Lu (¹⁷⁷Lu-Trastuzumab) at the specific activity of 200 MBq/mg. AGuIX® dissolved directly in 1 mL of water for injectable preparation (WFI) and stirred for 10 min at 25° C., then diluted in DMEM/F12 culture medium to achieve a concentration range of 1-10 mg/mL. ¹⁷⁷Lu-Trastuzumab was used to treat SK-OV-3 and A431 cells. Moreover, LUTATHERAC (¹⁷⁷Lu-DOTATATE, targeting sst2) was used to treat MiaPaca2 cells, and ¹²⁵I-TA99 mAb was used to treat B16F10 cells, targeting TYRP1/gp75 receptors. It is noteworthy that LUTATHERA@ is routinely used in the clinic for treating midgut neuroendocrine tumor patients (Strosberg et al., 2017; Strosberg et al., 2021).

### In Vivo

At day 14 following xenograft, mice were divided into different groups (n=8), and received the IP injections summarized in Table 1 below:

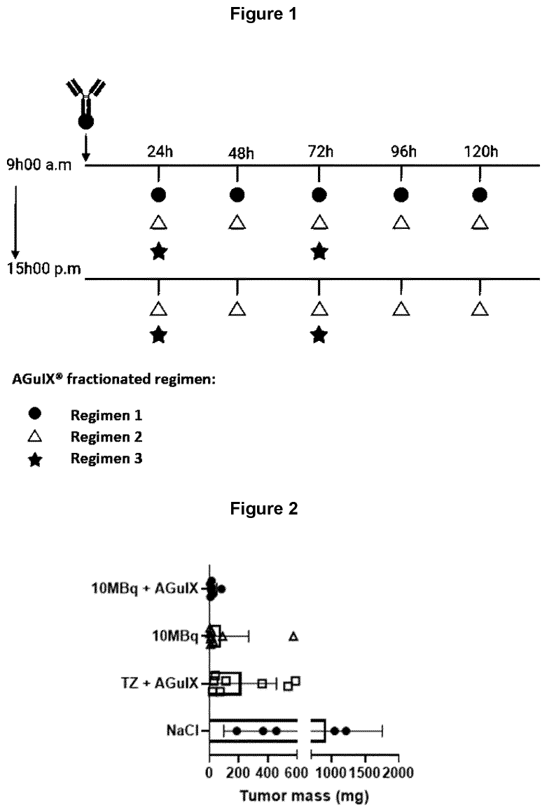

TABLE 1

| | |
|---|---|
| NaCl | 200 μl of saline solution |
| Trastuzumab + AGulX ® | Trastuzumab (equivalent of radiolabeled antibody |
| | injected to mice receiving the greater activity) in 200 μl |
| | of saline solution + 10 mg AGulX ® in 200 μl of saline |
| | solution. |
| Injected activities (IA) | 2.5, 5 or 10 MBq ¹⁷⁷Lu-Trastuzumab in 200 μl saline |
| | solution. 10 MBq is the maximal tolerated activity for |
| | nude mice. |
| IA + AGulX | 2.5, 5 or 10 MBq ¹⁷⁷Lu-Trastuzumab in 200 μl saline |
| | solution + 10 mg AGulX ® in 200 μl of saline solution. |
| AGulX ® fractionated | AGulX ® fractionated regimen as illustrated in FIG. 1. |
| regimen | Regimen 1 (5 × 4 mg): mice received a single injection |
| | of 4 mg AGulX in 200 μl saline solution for 5 consecutive |
| | days. |
| | Regimen 2 (10 × 2 mg): mice received two injections of |
| | 2 mg AGulX in 200 μl saline solution per day (separated |
| | with a 6 h time lapse) for 5 consecutive days. |
| | Regimen 3 (4 × 5 mg): mice received two injections of |
| | 5 mg AGulX in 200 μl saline solution per day (separated |
| | with a 6 h time lapse) 24 h and 72 h post-TRT. |

### ICP-MS

SK-OV-3-luc xenografts received 10 mg (7200 nmol Gd) of AGuIX®, tumors and organs of interest were recovered ex vivo 30 minutes, 6 h, 24 h and 48 h post-injection (n=3 mice per timepoint). Samples were analyzed by ICP-MS for gadolinium quantification.

### Biodistribution of Radiolabeled Antibody

Athymic female Swiss nude mice were intraperitoneally (IP) xenografted with 3×10⁶ SK-OV-3-luc cells. 14 days later, mice were IP injected with ¹⁷⁷Lu-labeled Trastuzumab. Tumors and organs were collected, weighed and radioactivity uptake measured by γ-counting. For each organ or tumor, percentage of injected activity per gram of tissue (% IA/g) were plotted.

### Results

### a 10 MBq Activity is Too Effective to Appreciate AGuIX® Radiosensitizing Effect.

The results shown in FIG. 2 demonstrates that the combination TRT+AGuIX®:
Decreased by a 97% total tumor mass (*** p=0.0006) compared to NaCl control.
Decreased by an 89% the total tumor mass (** p=0.006) compared to Trastuzumab+AGuIX®.
No significant differences were found when compared with TRT alone.

The Maximum Tolerated Activity (MTA) of ¹⁷⁷Lu-Trastuzumab (10MBq), showed a strong therapeutic efficacy, which does not allow to appreciate a potential radiosensitizing of AGuIX nanoparticles. In further experiments, ¹⁷⁷Lu-Trastuzumab activity were reduced to 5 and 2.5 MBq.

### An Activity of 5MBq ¹⁷⁷Lu-Trastuzumab Associated to 10 mg AGuIX® Showed a Radiosensitizing Trend

The results of FIG. 3 show that:
2.5 MBq ¹⁷⁷Lu-Trastuzumab+/−AGuIX® didn't show a significant efficacy when compared to Trastuzumab+AGuIX® control.
5 MBq ¹⁷⁷Lu-Trastuzumab+AGuIX® decreased by a 97% (*** p=0.0003) and a 94% (** p=0.001) the total tumor mass compared to NaCl and Trastuzumab+AGuIX® control respectively.
No significant differences were found when 5 MBq+AGuIX® was compared with TRT alone.

An injected activity of 5 MBq ¹⁷⁷Lu-Trastuzumab was established as working activity for further experiments. The lack of significant radiosensitizing effect could be related, besides to the high efficacy of TRT alone, to the NP uptake and retention duration into tumors. AGuIX® biodistribution was further performed on tumor-bearing animals to assess their uptake/elimination kinetics.

### AGuIX® Showed Fast Elimination and Insufficient Long-Term Tumors Retention.

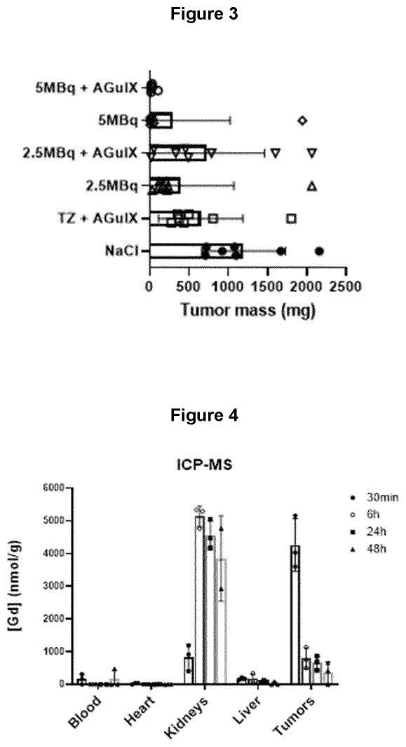

The results of FIG. 4 show a quick flush-out of the nanoparticles within tumors between 30 minutes and 6 h post-injection. This elimination kinetics is suitable when an External Beam of Radiation is combined with AGuIX®, in which irradiation arrives at a high dose rate in a flash manner. For a TRT approach, irradiation is performed at a lower dose rate but maintained over time. Therefore, a longer time of residence of the nanoparticle in tumors was desired. Altogether, these observations led to the design of 3 fractionated administration regimens of AGuIX® to be combined with TRT.

Fractionated Regimen of AGuIX® Significantly Increases Tumor Regression when Compared to TRT Alone.

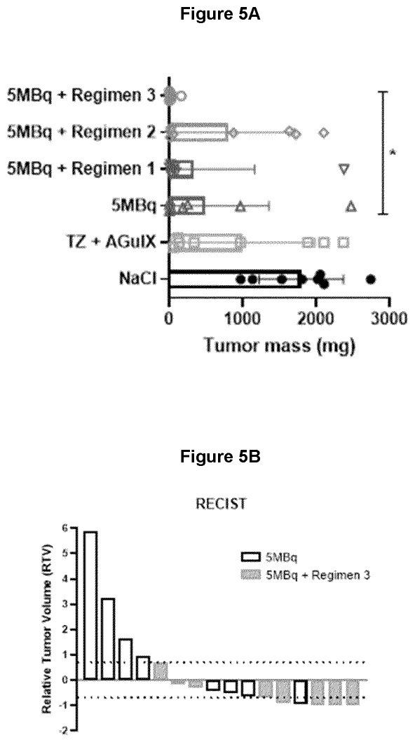

The results of FIG. 5A show a significant difference between TRT (5MBq) and the fractionated Regimen 3 (4×5 mg).

In order to compare responses to both treatments, the Response Evaluation Criteria In Solid Tumors (RECIST) criteria (Gengenbacher et al., 2017). was evaluated (FIG. 5B). Briefly, the RECIST based 3-categories method classifies drug responses into 3 categories: Complete response (CR), stable disease (SD) and progressive disease (PD) based on relative tumor volume, or RTV, at a later day relative to treatment initiation (OR: RTV≤0.65, PD: RTV≥1.35, SD: 0.65<RTV<1.35). The results are shown in FIG. 5B and Table 2 below:

TABLE 2

| RECIST | 5 MBq | 5 MBq + Regimen 3 |
|---|---|---|
| Complete | 1 | 5 |
| Response | | |
| Stable Disease | 4 | 3 |
| Progressive | 3 | 0 |
| Disease | | |

### In Vitro, AGuIX® Radiosensitizes Cancer Cells to Beta and Auger TRT.

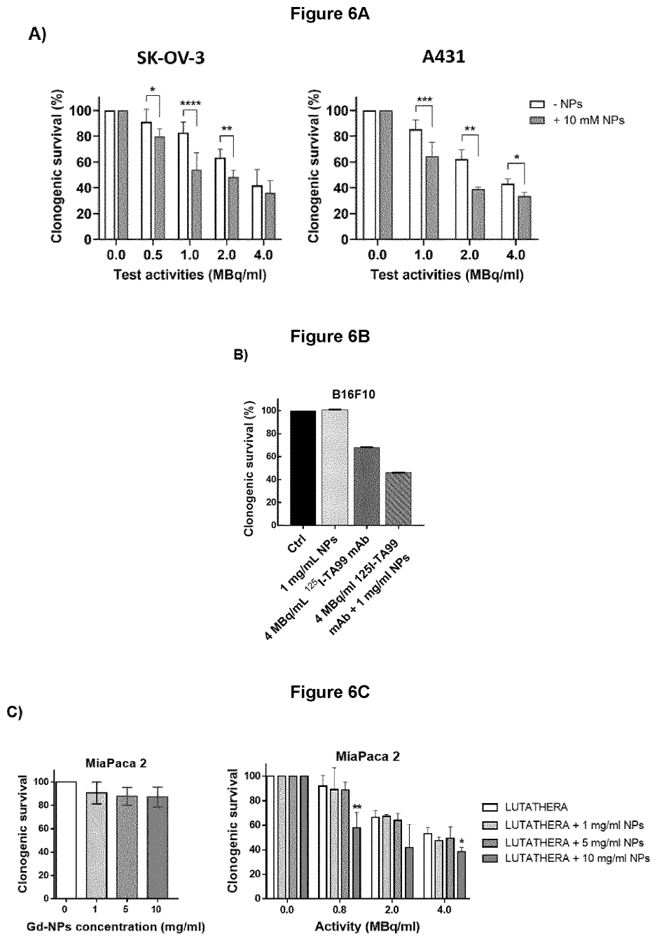

The results of FIG. 6 show:
FIG. 6A: Clonogenic cell survival of SK-OV-3 and A431 cells exposed to ¹⁷⁷Lu-Trastuzumab at 0.5, 1, 2 and 4 MBq/mL±10 mg/ml of AGuIX®.
FIG. 6B: Clonogenic cell survival of B16F10 cells exposed to 4 MBq/mL ¹²⁵I-TA99±1 mg/ml of AGuIX®.
FIG. 6C: Clonogenic cell survival of MiaPaca2 cells exposed to 0.8, 2 and 4MBq/mL of LUTATHERA®±1, 5 and 10 mg/ml of AGuIX®.

AGuIX® treatment alone did not produce a significant cytotoxic effect on any of the tested cell lines. A significant radiosensitizing effect was found when gynecologic cancer cell lines SK-OV-3 and A431 were incubated with ¹⁷⁷Lu-Tratuzumab in combination with 10 mg/mL of AGuIX® (FIG. 6A). Consistently, a radiosensitizing effect was found when B16F10 murine melanoma cells were exposed to 4MBq/mL ¹²⁵I-TA99 in the presence of 1 mg/ml of AGuIX® (FIG. 6B). Likewise, a significant radiosensitizing effect was shown when MiaPaca2 pancreatic cancer cells were treated with LUTATHERA® in the presence of 10 mg/ml of AGuIX® (FIG. 6C).

### Conclusion

The in vitro and in vivo results demonstrate the radiosensitization and enhanced therapeutic efficacy of the therapeutic radiopharmaceutical when administered in combination with AGuIX®, while reducing the total injected activity. This result could have a major impact for patients by reducing radiation-induced toxicity.

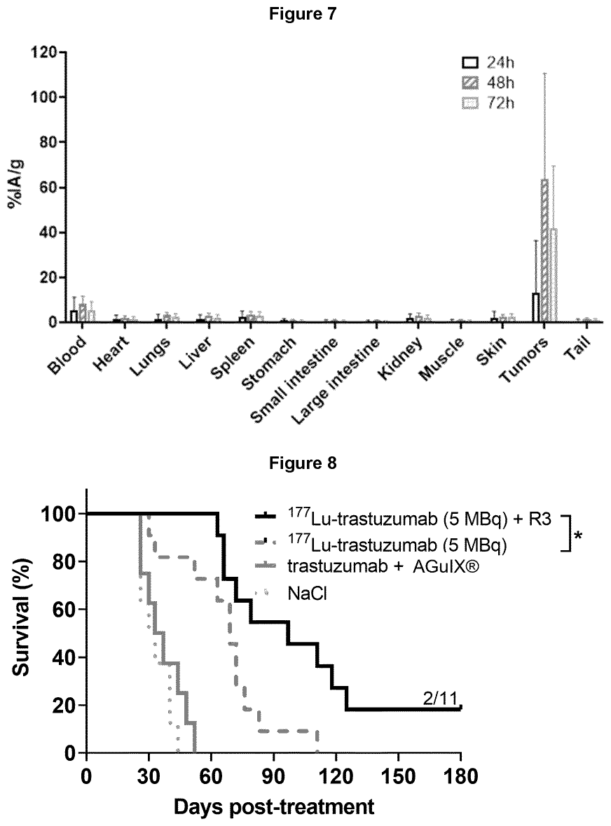

Besides, the inventors have generated biodistribution data to follow the fate of the radiolabeled antibody ¹⁷⁷Lu-Trastuzumab (FIG. 7) and of the AGuIX® nanoparticles in the tumor. The monitoring of the NPs in the tumor was ensured by ICPMS measurements while that of the radiolabeled antibody was ensured by measurement of the radioactivity in the tumor. The results demonstrate that:
the incorporation of the radiolabeled antibody in the tumor increases between 0 and 48 h and then decreases.
the incorporation of the nanoparticles (NPs) in the tumor is more rapid. It increases in 30 min and then decrease.

Therefore, by injecting the NPs between T24 h, T30 h, T72 h, and T78 h after the antibody injection, at a given time the NPs and the antibody are in the tumor at the same time, i.e. colocalize.

### Example 2: Animal Survival Upon Regimen 3

The animal survival upon the administration of Regimen 3 as described in Example 1 was assayed.

### Material and Methods

In this second set of experiments, mice received two injections of 5 mg AGuiX® in 200 μl saline solution per day (separated with a 6 h time lapse) 24 h and 72 h post-TRT. The survival of mice was monitored by bioluminescence measurement for 130 days post-treatment. Kaplan-Meier survival estimates were calculated from the xenograft date to the date of the event of interest (i.e., bioluminescence of 4×10¹⁰ photons/s) and compared with the log-rank test.

### Results

The results are shown on FIG. 8.

The Kaplan Meyer survival curves established confirm that Regimen 3 (R3) led to the strongest improvement in median survival (FIG. 8). Specifically, [¹⁷⁷Lu-trastuzumab+R3] led to a significant increase in survival (median survival=97 days; 2 mice cured over 11) compared with NaCl (median survival=30 days; **** p<0.0001), trastuzumab+AGuiX® (median survival=33 days; **** p<0.0001), and 5MBq [¹⁷⁷Lu-trastuzumab alone (median survival=69 days; * p=0.016).

### Example 3: Co-Localization of High-Z Containing NPs According to the Invention and Lysosomes

### Material and Methods

To assess the localization of nanoparticles relative to mitochondria and lysosomes, SK-OV-3-luc cells were incubated with AGuIX®-AF488 (i.e. AGuIX® functionalized with Alexa Fluor dye) for 18 h followed by incubation with Mitotracker™ Red CM-H 2Xros (M7513, Thermofisher), or Lysotracker™ Red DND-99 (L7528, Thermofisher) for 45 min.

### Results

Using the latter methodology, it was shown that AGuiX® co-localize with lysosomes, but not with mitochondria (data not shown). These results were confirmed by Transmission Electron Microscopy (TEM) imaging.

### Example 4: Role of Iron in the Radiosensitizing Effects of the High-Z Containing NPs According to the Invention

### Materials and Methods

Cells were seeded in 6-well plates at a density of 100-300 cells/well. The following day, cells were incubated with increasing activities (0-4 MBq/mL) of ¹⁷⁷Lu-trastuzumab combined or not with 10 mg/mL AGuiX® for 18 h and in the presence of 100 μM Deferiprone (DFP) (Selleck Chemicals). Then, culture medium was removed, cells were washed twice with 1×PBS, and fresh medium was added and cells kept for clonogenic survival as described above. Three independent experiments were performed in triplicate.

### Results

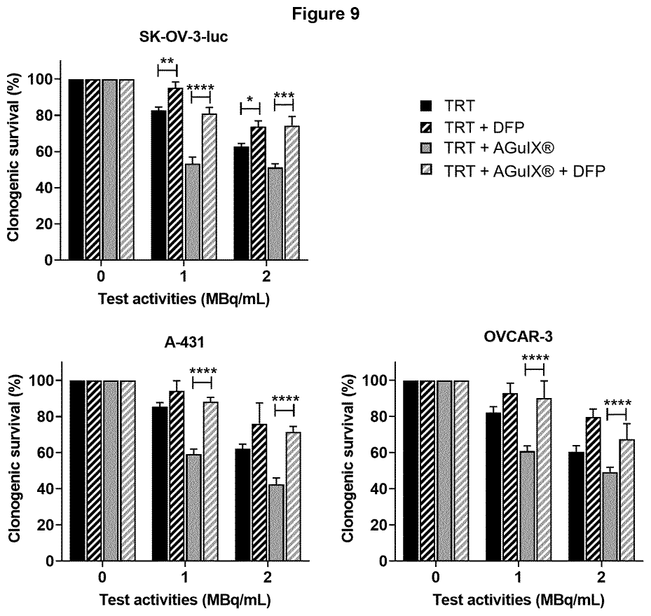

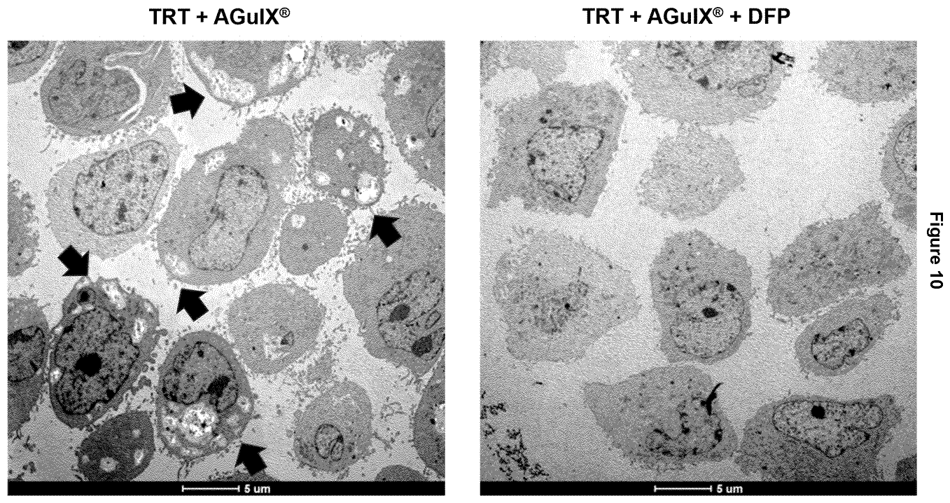

The results are shown on FIG. 9 and FIG. 10.

The results on FIG. 9 show that the sensitizing effect of AGuiX® to TRT is abolished in the presence of deferiprone (an iron chelator), as measured by the clonogenic survival of treated cells. Lysosomes contain iron participating to reactive oxygen species (ROS) formation via the Fenton reaction. The process is exacerbated when AGuiX® NPs are combined with TRT and leads to lysosomal disruption. The oxidative process leads to lysosomes disruption as showed by their decreased number and with cytoplasmic pH decrease (data not shown). Iron released from disrupted lysosomes also generate waves of ROS in the cytoplasm leading to DNA damage as shown by a rise in micronuclei formation (data not shown). The role of ROS in the sensitizing effect of AGuiX® to TRT is confirmed by the use of ROS scavengers (DMSO, NAC) and antioxidant enzymes (Catalase). Consequently, a dramatic lipid peroxidation is observed and suggest a potential role of ferroptosis in AGuIX®-mediated toxicity.

Altogether, these results demonstrate that the combination TRT+AGuIX® efficacy is mediated by ferroptosis.

These results were confirmed by TEM images of cells treated with TRT and AGuiX® in the presence or not of deferiprone (FIG. 10).

The TEM micrographs as depicted in FIG. 10 show SKOV3 cells 48 h after treatment with 1MBq/mL of ¹⁷⁷Lu-Trastuzumab+10 mg/mL AGuIX® in the presence of the iron chelator Deferiprone (DFP). Cytoplasm dissolution/necrosis-like features with yellow arrows. The left panel of FIG. 10 shows the outcome of TRT+AGuiX® treatment 48 h post-incubation and the right panel of FIG. 10 shows the same treatment in the presence of DFP. Cytoplasmic vacuolization (arrow) is abolished in the presence of the iron chelator.

Altogether, the TEM micrographs of FIG. 10 show that the TRT+AGuIX® combination leads to dramatic ultrastructural modifications as observed, characterized by a massive cytoplasmic vacuolization followed by an apparent cytoplasmic dissolution and the accumulation of autophagosomes and damaged undigested cell components. It was thus shown that iron chelation increases the TRT+AGuIX®-treated cell survival, allows the recovery of lysosomal integrity, and reverses the ultrastructural modifications mentioned above.

### BIBLIOGRAPHY

Aarts F, Hendriks T, Boerman O C, Koppe M J, Oyen W J G, Bleichrodt R P. A Comparison Between Radioimmunotherapy and Hyperthermic Intraperitoneal Chemotherapy for the Treatment of Peritoneal Carcinomatosis of Colonic Origin in Rats. Ann Surg Oncol.; 14(11):3274-82 (2007).
Aarts F, Bleichrodt R P, de Man B, Lomme R, Boerman O C, Hendriks T. The Effects of Adjuvant Experimental Radioimmunotherapy and Hyperthermic Intraperitoneal Chemotherapy on Intestinal and Abdominal Healing after Cytoreductive Surgery for Peritoneal Carcinomatosis in the Rat. Ann Surg Oncol.; 15(11):3299-307. (2008).
Andersson H, Elgqvist J, Horvath G, Hultborn R, Jacobsson L, Jensen H, et al. Astatine-211-labeled antibodies for treatment of disseminated ovarian cancer: an overview of results in an ovarian tumor model. Clin Cancer Res.; 9(10 Pt 2):3914S-21S. (2003).
Alvarez R D, Huh W K, Khazaeli M B, Meredith R F, Partridge E E, Kilgore L C, et al. A Phase I study of combined modality (90) Yttrium-C C49 intraperitoneal radioimmunotherapy for ovarian cancer. Clin Cancer Res.; 8(9):2806-11 (2002).
Elgqvist J, Andersson H, Back T, Hultborn R, Jensen H, Karlsson B, et al. Therapeutic efficacy and tumor dose estimations in radioimmunotherapy of intraperitoneally growing OVCAR-3 cells in nude mice with (211) At-labeled monoclonal antibody MX35. J Nucl Med.; 46(11):1907-15 (2005).
Epenetos A A, Hird V, Lambert H, Mason P, Coulter C. Long term survival of patients with advanced ovarian cancer treated with intraperitoneal radioimmunotherapy. Int J Gynecol Cancer.; 10(s1):44-6 (2000).
Gengenbacher, N., Singhal, M. & Augustin, H. Preclinical mouse solid tumour models: status quo, challenges and perspectives. Nat Rev Cancer 17, 751-765 (2017).
Goodman M D, McPartland S, Detelich D, Saif M W. Chemotherapy for intraperitoneal use: a review of hyperthermic intraperitoneal chemotherapy and early post-operative intraperitoneal chemotherapy. J Gastrointest Oncol.; 7(1):45-57 (2016).
Hird V, Maraveyas A, Snook D, Dhokia B, Soutter W, Meares C, et al. Adjuvant therapy of ovarian cancer with radioactive monoclonal antibody. Br J Cancer.; 68(2):403-6 (1993).
Meredith R F, Buchsbaum D J, Alvarez R D, LoBuglio A F. Brief Overview of Preclinical and Clinical Studies in the Development of Intraperitoneal Radioimmunotherapy for Ovarian Cancer. Clin Cancer Res.; 13(18):5643s-5s (2007).
Koppe M J, Bleichrodt R P, Oyen W J G, Boerman O C. Radioimmunotherapy and colorectal cancer. British Journal of Surgery.; 92(3):264-76 (2005)
Milenic D E, Garmestani K, Brady E D, Albert P S, Ma D, Abdulla A, et al. Targeting of HER2 Antigen for the Treatment of Disseminated Peritoneal Disease. Clin Cancer Res.; 10(23):7834-41 (2004).
Müller C, Zhernosekov K, Koster U, Johnston K, Dorrer H, Hohn A, et al. A Unique Matched Quadruplet of Terbium Radioisotopes for PET and SPECT and for α- and β-Radionuclide Therapy: An In Vivo Proof-of-Concept Study with a New Receptor-Targeted Folate Derivative. J Nucl Med.; 53(12) :1951-9. (2012)
Sgouros G, Bodei L, McDevitt M R, Nedrow J R. Radiopharmaceutical therapy in cancer: clinical advances and challenges. Nat Rev Drug Discov.; 19(9):589-608 (2020)
Pouget J P, Navarro-Teulon I, Bardies M, Chouin N, Cartron G, Pelegrin A, et al. Clinical radioimmunotherapy—the role of radiobiology. Nat Rev Clin Oncol.; 8(12):720-34 (2011)
Pouget J P, Lozza C, Deshayes E, Boudousq V, Navarro-Teulon I. Introduction to Radiobiology of Targeted Radionuclide Therapy. Front Med.; 17; 2:12 (2015)
Seidl C, Zöckler C, Beck R, Quintanilla-Martinez L, Bruchertseifer F, Senekowitsch-Schmidtke R. ¹⁷⁷Lu-immunotherapy of experimental peritoneal carcinomatosis shows comparable effectiveness to ²¹³Bi-immunotherapy, but causes toxicity not observed with 213Bi. Eur J Nucl Med Mol Imaging.; 38(2):312-22. (2011)
Strosberg J, E1-Haddad G, Wolin E, Hendifar A, Yao J, Chasen B, et al. Phase 3 Trial of ¹⁷⁷Lu-Dotatate for Midgut Neuroendocrine Tumors. N Engl J Med.; 376(2):125-35 (2017)
Strosberg J, Leeuwenkamp O, Siddiqui MohdK. Peptide receptor radiotherapy re-treatment in patients with progressive neuroendocrine tumors: A systematic review and meta-analysis. Cancer Treat Rev.; Vol. 93:102141 (2021)
Sugarbaker P H. Comprehensive management of peritoneal surface malignancy using cytoreductive surgery and perioperative intraperitoneal chemotherapy: the Washington Cancer Institute approach. Expert Opinion on Pharmacotherapy. Expert Opin Pharmacother; 10(12):1965-77 (2009).
Verheijen R H, Massuger L F, Benigno B B, Epenetos A A, Lopes A, Soper J T, et al. Phase III Trial of Intraperitoneal Therapy With Yttrium-90-Labeled HMFG1 Murine Monoclonal Antibody in Patients With Epithelial Ovarian Cancer After a Surgically Defined Complete Remission. JCO.; 24(4):571-8 (2006).

1. A method of treating a tumor by radiopharmaceutical therapy, in a subject in need thereof, the method comprising a combined administration of an efficient amount of high-Z element containing nanoparticles and of an efficient amount of a radionuclide containing therapeutic radiopharmaceutical, wherein the high-Z element containing nanoparticles contain an element with an atomic Z number higher than 40, and wherein said nanoparticles have a mean hydrodynamic diameter of 20 nm or less.

2. The method according to claim 1, wherein the nanoparticles enhance the therapeutic efficacy of said radiopharmaceutical.

3. The method according to claim 1, wherein the high-Z element is selected among the heavy metals.

4. The method according to claim 1, wherein the radionuclide is selected from the group consisting of ¹⁷⁷Lu, ¹⁶¹Tb, ¹⁸⁶Re, ¹³¹I, ⁹⁰Y, ²²⁵Ac/²¹³Bi, ²²³Ra, ²¹²Pb/²¹²Bi, ²²⁷Th, ²¹¹At, ⁹⁷Ru, ¹⁰³Pd, ⁶⁷Ga, ^{195m}Pt, ^{193m}Pt, ¹²⁵I, ¹¹¹In, and mixtures thereof.

5. The method according to claim 1, wherein the efficient amount of radiopharmaceutical is comprised between 0.5.MBq and 100 GBq.

6. The method according to claim 1, wherein the radionuclide is linked to a cancer-targeting moiety.

7. The method according to claim 10, wherein the cancer-targeting moiety is an antibody, a peptide or a small-molecule ligand.

8. The according to claim 10, wherein the cancer-targeting moiety is selected from the groups consisting of
an anti-HER2 antibody an anti-EGFR antibody, an anti-CD20 antibody, an anti-CD33 antibody, an anti-CD37 antibody, an anti-AMHRII antibody, or an anti-TYRP1/gp75 antibody,
a somatostatin analog and
a PSMA small-molecule ligand.

9. The method according to claim 1, wherein the therapeutic radiopharmaceutical is selected from the group consisting of an ¹⁷⁷Lu-anti-HER2 antibody, an ¹⁷Lu-somatostatin analog, a ¹⁷⁷Lu-PSMA ligand, an ⁹⁰Y-anti-CD20 antibody, an ²¹²Pb-anti-HER2 antibody, and an ¹⁷⁷Lu-anti CD37 antibody.

10. The according to claim 1, wherein the therapeutic radiopharmaceutical consists of ¹³¹I or ²²³Ra.

11. The method according to claim 1, wherein the nanoparticles are administered to the subject in a fractionated dose regimen.

12. The method according to claim 1, wherein the fractionated dose regimen of nanoparticles comprises 2 to 10 fractionated doses of nanoparticles for each dose of therapeutic radiopharmaceutical administered to the subject.

13. The method according to claim 1, wherein the subject is a subject that cannot receive the standard effective dose of a radiopharmaceutical therapy.

14. The method according to claim 1, wherein the tumor is a radioresistant tumor.

15. The method according to claim 1, wherein the tumor is selected from the group consisting of peritoneal tumors including primary peritoneal tumors and secondary peritoneal tumors, neuroendocrine tumors including gastroenteropancreatic neuroendocrine tumors and pheochromocytoma or paragangliomas (PPGLs), prostate tumor, neuroblastoma, meningioma, lymphoma, Merkel cell carcinoma, breast tumor, renal cell tumor, and salivary gland carcinoma.

16. The method according to claim 1, wherein the nanoparticles contain an element with an atomic Z number higher than 50.

17. The method according to claim 1, wherein the nanoparticles have a mean hydrodynamic diameter between 2 and 8 nm.

18. The method according to claim 3, wherein nanoparticles have a mean hydrodynamic diameter between 2 and 8 nm.

19. The method according to claim 6, wherein the high-Z element is selected from the group consisting of Au, Ag, Pt, Pd, Sn, Ta, Zr, Tb, Tm, Ce, Dy, Er, Eu, La, Nd, Pr, Lu, Yb, Bi, Hf, Ho, Pm, Sm, In, and Gd, and mixtures thereof.
